(19) United States
(12) Patent Application Publication
(10) Pub. No.: US 2016/0058855 A1
(43) Pub. Date: Mar. 3, 2016

(54) HIGH PURITY OVARIAN CANCER STEM CELLS FOR ACTIVE AUTOLOGOUS IMMUNE THERAPY

(71) Applicant: NEOSTEM ONCOLOGY, LLC, Irvine, CA (US)

(72) Inventors: Andrew N. CORNFORTH, Irvine, CA (US); Gabriel NISTOR, Irvine, CA (US)

(73) Assignee: NEOSTEM ONCOLOGY, LLC, Irvine, CA (US)

(21) Appl. No.: 14/775,313

(22) PCT Filed: Mar. 10, 2014
(86) PCT No.: PCT/US2014/022494

§ 371 Date: Sep. 11, 2015

(30) Foreign Application Priority Data
Aug. 6, 2013 (US) PCTUS2013 053850

(51) Int. Cl. A61K 39/00; A61K 35/13; C12N 5/0784

(52) U.S. Cl. CPC A61K 39/0011; C12N 5/0639; A61K 35/13; C12N 2501/115; A61K 2039/5154; A61K 2039/55522; A61K 2039/545

(57) ABSTRACT

The disclosure provides cancer stem cells, for use in stimulating immune response against a cancer, such as ovarian carcinoma. Methods for preparing and purifying the cancer stem cells are provided.

### CROSS-REFERENCE TO RELATED APPLICATIONS

This application claims the benefit under 35 U.S.C. §119(e) to U.S. Provisional Patent Application 61/778,225 filed Mar. 12, 2013. The present application is also a continuation-in-part of International Application PCT/US2013/053850 filed Aug. 6, 2013, which claims the benefit under 35 U.S.C. §119(e) to U.S. Provisional Patent Applications 61/683,477 filed Aug. 15, 2012 and 61/718,643 filed Oct. 25, 2012. The entire contents of all of which are incorporated by reference herein.

### FIELD OF THE DISCLOSURE

The present disclosure relates to ovarian carcinoma stem cells, immunogenic compositions derived therefrom, and methods of making and using same.

### BACKGROUND OF THE DISCLOSURE

Ovarian cancer is thought to have the origins in the epithelia lining the ovaries, hence the adenocarcinoma histological characteristics. Ovarian cancer stem cells have been previously described as a subpopulation or “side population” (SP) that is selected from the main population (MP) in the SKOV3 and A224 cell lines, expressing stem cell marker genes (Oct4 and Nanog), transporter genes (ABCG2, ABCC4, ABCB1) and CD markers (CD44, CD24, CD177), with potential to differentiate into cancers, with different histologies, suggesting the pluripotent character of stem cells. Isolation of such cells was accomplished using the exclusion of the fluorescent DNA-binding dye Hoechst 33342

Specific cancer stem cell populations can be the origin of neoplasms and can be a source of recurrence of a cancer that had been treated. Also, subpopulations of cancer stem cells in a tissue can, when exposed to certain signals, restart the growth cycle and produce cells that can reestablish the tumor. The cancer stem cell niche is dormant until proper signaling triggers the re-entry in the proliferation cycle. Re-entry signals can originate from local events such as trauma, cell damage, microorganism aggression (viral, bacterial or fungal), or mediated by local growth factors, cytokines or intercellular communication. Also, hormones can modulate stem cells in tissue-specific niches. Defects or mutations of the stem cell niche can result in perturbation of the above functions. Neoplasms can result from such perturbations, and these include random mutations that influence control over the cell cycle. Mutations leading to cancer vary from individual to individual. Such variability is observed between those who suffer from one type of cancer, such as one breast cancer patient versus another breast cancer patient, as well as between different types of cancer, such as ovarian cancer versus melanoma. In ovarian cancer, damage to the TP53 gene has been identified in the majority of the cases, however this mutation is not always reflected in the tumor cell phenotype.

Inconsistent markers are also associated in various proportions with the tumor cells and used more or less successfully to trigger an immune-response. Such therapies using tumor-associated antigens employ various proteins or peptides such as CA125, Her-2, Muc-1, Neu, NY-ESO-1, or tumor derived heat shock proteins (HSP).

Autologous immune therapies employ a patient's own tumor tissue to sensitize the immune system and attach the cancerous cells. Lysates or whole cell approaches are administrated alone or with adjuvants have been used to enhance the immune response to tumors.

### SUMMARY

Disclosed herein are ovarian carcinoma (OV) cancer stem cells (CSC), OV-CSC cell lines, and immunogenic compositions comprising OV-CSC-loaded dendritic cells for the treatment of ovarian carcinoma.

Specifically provided herein is an immunogenic composition comprising dendritic cells activated ex vivo by tumor antigens derived from the population of purified ovarian carcinoma (OV) cancer stem cells (CSC) disclosed herein. In one embodiment, the tumor antigens comprise cell extracts of the OV-CSC. In another embodiment, the tumor antigens comprise lysates of the OV-CSC. In another embodiment, the tumor antigens comprise intact OV-CSC. In another embodiment, the tumor antigens comprise messenger RNA transfected into the dendritic cells ex vivo.

In another embodiment, the intact OV-CSC are rendered non-proliferative. In another embodiment, the intact OV-CSC are rendered non-proliferative by irradiation. In yet another embodiment, the intact OV-CSC are rendered non-proliferative by exposure of the OV-CSC to a nuclear or protein cross-linking agent.

In one embodiment, the immunogenic composition further comprises a pharmaceutically acceptable carrier and/or excipient. In another embodiment, the immunogenic composition further comprises an adjuvant. In another embodiment, the adjuvant is granulocyte macrophage colony stimulating factor.

In yet another embodiment, immunogenic composition comprises activated dendritic cells and OV-CSC. In another embodiment, the OV-CSC are in the form of OV-CSC spheroids, early OV-CSC, mixed OV-CSC, or EMT-OV-CSC.

Also provided is a method of treating ovarian carcinoma in a subject in need thereof, comprising administration of an immunogenic composition disclosed herein to the subject. In one embodiment, the immunogenic composition is administered in a plurality of doses, each dose comprising about 5-20×10⁶ cells. In another embodiment, the dose comprises about 10×10⁶ cells. In another embodiment, the dose is administered weekly for 2-5 doses, followed by monthly for 3-6 doses. In yet another embodiment, the subject receives from 6-10 doses of immunogenic composition.

Also provided is the use of an immunogenic composition disclosed herein, an OV-CSC disclosed herein, or an OV-CSC cell line disclosed herein in the manufacture of a medicament for the treatment of ovarian carcinoma.

Also provided is the use of an immunogenic composition disclosed herein, an OV-CSC disclosed herein, or an OV-CSC cell line disclosed herein for the treatment of ovarian carcinoma.

Further provided herein is method for preparing a population of ovarian carcinoma (OV) cancer stem cells (CSC), the method comprising: acquiring a sample of OV; dissociating the cells of the sample, and in vitro culturing the dissociated cells in a defined medium on a non-adherent substrate, wherein the defined medium is serum free and is supplemented with at least one growth factor that acts through the mitogen activated protein kinase (MAPK) pathway, thereby forming OV-CSC spheroids; wherein at least 80% of the cells in the OV-CSC spheroid population express two or more of the biomarkers EpCAM, CA-125, MUC-1, CD117, He-4, ALDH, CD133, CD24, and Ki-67. In another embodiment, at least 80% of the cells in the OV-CSC spheroid population further express one or more of the biomarkers CA19-9, HER2/neu, NCAM, ganglioside CD2, estrogen receptor alpha, vimentin, CK8, CK18, AFP, testosterone, TGFβR, EGFR, TAG-72, CD46, CD44, ABCG2, Slug/Snail, nestin, and TP53. In another embodiment, at least 90% of the cells in the OV-CSC spheroid population express two or more of the biomarkers EpCAM, CA-125, MUC-1, CD117, He-4, ALDH, CD133, CD24, and Ki-67.

In another embodiment, the method further comprises culturing the OV-CSC spheroids in a defined medium on an adherent substrate, wherein the defined medium is serum free and is supplemented with at least one growth factor that acts through the MAPK pathway, thereby forming a population of early OV-CSC, wherein at least 80% of the cells in the early OV-CSC population express two or more of the biomarkers EpCAM, CD133, CD44, Nanog, Sox2, Oct3/4, CD17, and Ki-67. In another embodiment, at least 80% of the cells in the early OV-CSC population further express one or more of the biomarkers CA-125, MUC-1, TGFβR, and CD24. In yet another embodiment, at least 90% of the cells in the early OV-CSC population express two or more of the biomarkers EpCAM, CD133, CD44, Nanog, Sox2, Oct3/4, CD17, and Ki-67.

In another embodiment, the method further comprises culturing the OV-CSC spheroids in a defined medium on an adherent substrate, wherein the defined medium contains serum, thereby forming a population of mixed OV-CSC, wherein at least 80% of the cells in the mixed OV-CSC population express two or more of the biomarkers EpCAM, CA-125, MUC-1, CD117, CK8, CK18, and Ki-67. In another embodiment, the defined medium further comprises at least one growth factor that acts through the MAPK pathway. In yet another embodiment, the defined medium is a low calcium defined medium. In another embodiment, at least 80% of the cells in the mixed OV-CSC population further express one or more of the biomarkers CA19-9, HER2/neu, NCAM, ganglioside CD2, estrogen receptor alpha, testosterone, TGFβR, EGFR, TAG-72, CD46, He-4, ALDH, CD133, CD44, ABCG2, nestin, and TP53. In yet another embodiment, at least 90% of the cells in the mixed OV-CSC population express two or more of the biomarkers EpCAM, CA-125, MUC-1, CD117, CK8, CK18, and Ki-67.

In another embodiment, the method further comprises culturing the OV-CSC spheroids in a defined medium on an adherent substrate, wherein the defined medium contains serum and is supplemented with at least one growth factor that acts through the MAPK pathway, thereby forming a population of epithelial to mesenchymal transitioned (EMT)-OV-CSC, wherein at least 80% of the cells in the EMT-OV-CSC population express two or more of the biomarkers NCAM, Slug/Snail, CD24, and Twist. In another embodiment, at least 80% of the cells in the EMT-OV-CSC population further express one or more of the biomarkers CA-125, MUC-1, CD133, Nanog, CD117, N-cadherin, CD44, and vimentin. In another embodiment, at least 90% of the cells in the EMT-OV-CSC population express two or more of the biomarkers NCAM, Slug/Snail, CD24, and Twist.

In another embodiment, the method further comprises culturing the OV-CSC spheroids, the mixed OV-CSC, or the EMT-OV-CSC in a defined medium on an adherent substrate, wherein the defined medium is serum free and is supplemented with at least one growth factor that acts through the MAPK pathway, thereby forming a population of early OV-CSC, wherein at least 80% of the cells in the early OV-CSC population express two or more of the biomarkers EpCAM, CD133, CD44, Nanog, Sox2, Oct3/4, CD17, and Ki-67. In another embodiment, at least 80% of the cells in the early OV-CSC population further express one or more of the biomarkers CA-125, MUC-1, TGFβR, and CD24. In yet another embodiment, at least 90% of the cells in the early OV-CSC population express two or more of the biomarkers EpCAM, CD133, CD44, Nanog, Sox2, Oct3/4, CD17, and Ki-67.

In another embodiment, the method further comprises culturing the OV-CSC spheroids, the early OV-CSC, or EMT-OV-CSC in a defined medium on an adherent substrate, wherein the defined medium contains serum and is supplemented with at least one growth factor that acts through the MAPK pathway, thereby forming a population of mixed OV-CSC, wherein at least 80% of the cells in the mixed OV-CSC population express two or more of the biomarkers AFP, CK7, CK19, EpCAM, E-cadherin, Nanog, FoxA2 HNF4a, and ABCG2. In another embodiment, the defined medium further comprises at least one growth factor that acts through the MAPK pathway. In yet another embodiment, the defined medium is a low calcium defined medium. In another embodiment, at least 80% of the cells in the mixed OV-CSC population further express one or more of the biomarkers CA19-9, HER2/neu, NCAM, ganglioside CD2, estrogen receptor alpha, testosterone, TGFβR, EGFR, TAG-72, CD46, He-4, ALDH, CD133, CD44, ABCG2, nestin, and TP53. In yet another embodiment, at least 90% of the cells in the mixed OV-CSC population express two or more of the biomarkers EpCAM, CA-125, MUC-1, CD117, CK8, CK18, and Ki-67.

In another embodiment, the method further comprises culturing the OV-CSC spheroids, the early OV-CSC, or mixed OV-CSC in a defined medium on an adherent substrate, wherein the defined medium contains a serum source and is supplemented with at least one growth factor that acts through the MAPK pathway, thereby forming a population of EMT-OV-CSC, wherein at least 80% of the cells in the EMT-OV-CSC population express two or more of the biomarkers NCAM, Slug/Snail, CD24, and Twist. In another embodiment, at least 80% of the cells in the EMT-OV-CSC population further express one or more of the biomarkers CA-125, MUC-1, CD133, Nanog, CD117, N-cadherin, CD44, and vimentin. In another embodiment, at least 90% of the cells in the EMT-OV-CSC population express two or more of the biomarkers NCAM, Slug/Snail, CD24, and Twist.

In one embodiment, the defined media is any media described in Table 2, any media from a combination of Table 2 and Table 3, any media from a combination of Table 2, Table 3, and Table 4, or any media from a combination of Table 2 and Table 4.

In one embodiment, the growth factor is one or more of fibroblast growth factor (FGF), epidermal growth factor (EGF), or activin A. In another embodiment, the FGF is basic FGF (bFGF). In another embodiment, the defined medium is not supplemented with activin A. In yet another embodiment, the defined medium is supplemented with an agonist of activin A, in an amount effective to prevent spontaneous differentiation of OV stem cells. In another embodiment, the media comprises an antagonist of activin A, and the antagonist is follistatin or an antibody that specifically binds to activin A.

In another embodiment, the medium is not supplemented with an antioxidant. In another embodiment, the antioxidant is superoxide dismutase, catalase, glutathione, putrescine, or β-mercaptoethanol. In yet another embodiment, the medium is supplemented with glutathione.

In another embodiment, the adherent substrate is configured to adhere to, and optionally to collect, anchorage dependent cells, such as fibroblasts. In another embodiment, the non-adherent substrate is an ultralow adherent polystyrene surface. In yet another embodiment, the adherent substrate comprises a surface coated with a protein rich in RGD tripeptide motifs.

Also provided is a population of purified OV-CSC cells prepared by any of the method disclosed herein. In certain embodiments, the OV-CSC are OV-CSC spheroids, early OV-CSC, mixed OV-CSC, or EMT-OV-CSC.

Also provided is an OV-CSC cell line prepared by any of the method of disclosed herein. In certain embodiments, the OV-CSC are OV-CSC spheroids, early OV-CSC, mixed OV-CSC, or EMT-OV-CSC.

Also provided is method of stimulating an immune response against ovarian carcinoma in a subject in need thereof, comprising administration of an immunogenic composition disclosed herein, OV-CSC cells disclosed herein, or an OV-CSC cell line disclosed herein.

Also provided is use of OV-CSC cells disclosed herein, or a OV-CSC cell line disclosed herein in the manufacture of a medicament for the treatment of ovarian carcinoma.

Also provided is use of OV-CSC cells disclosed herein or a OV-CSC cell line disclosed herein for the treatment of ovarian carcinoma.

### BRIEF DESCRIPTION OF THE DRAWINGS

FIG. 1 is a flow chart of the process of isolation, expansion, and harvest of ovarian carcinoma (OV) cancer stem cells (OV-CSC) from an excised tumor (solid boxes and arrows) or from a small sample such as a needle biopsy or peritoneal lavage (dashed boxes and arrows) into spheroids. After generation of spheroids, the pathway of producing OV-CSC subpopulations is a common pathway

FIG. 2 depicts an established tumor cell line (OVCAR3) that produce irregular cell agglomerates in non-adherent conditions, thus demonstrating a higher degree of differentiation (phase contrast 20×)

FIG. 3 depicts patient-derived ovarian cancer stem cells that produce typical spheres in non-adherent conditions, thus demonstrating the presence and expansion of the cancer stem cells.

FIG. 4A-D depicts a patient-derived ovarian cancer stem cell culture labeled for cancer stem cell biomarkers EpCAM and NCAM (FIG. 4A). The cells were plated in 5% serum containing media, 10 ng/mL bFGF, and 10 ng/mL EGF. FIG. 4B depicts a red channel image of the cells of FIG. 4A reflecting the NCAM-positive cells. FIG. 4C depicts a green channel image of the cells of FIG. 4A demonstrating loss of EpCAM in a phenomenon associated with an epithelial to mesenchymal transition (EMT) of the cells. FIG. 4D depicts a blue channel image for bisbenzimide nuclear staining.

FIG. 5A-D depicts a patient-derived ovarian stem cancer cell culture labeled for EMT biomarkers Slug/Snail and CD117 (FIG. 5A). The cells were plated in 5% serum containing media, 10 ng/mL bFGF, and 10 ng/mL EGF. FIG. 5B depicts a red channel image indicating that the majority of the cells of FIG. 5A are positive for Slug/Snail. FIG. 5C depicts a green channel image indicating that the cells of FIG. 5A are positive for CD117. FIG. 5D depicts a blue channel image for bisbenzimide nuclear staining.

FIG. 6A-C depicts a patient-derived ovarian cancer stem cell culture labeled for cancer stem cell biomarker nestin (FIG. 6A). The cells were plated in 5% serum containing media, 10 ng/mL bFGF, and 10 ng/mL EGF. FIG. 6B depicts a red channel image indicating that the majority of cells are positive for nestin. This phenomenon is associated with EMT. FIG. 6C depicts a blue channel image for bisbenzimide nuclear staining.

FIG. 7 depicts a patient-derived ovarian cancer stem cell culture in a media containing 5% FBS on gelatin (phase contrast, 10×)

FIG. 8 depicts a patient-derived ovarian cancer stem cell culture in a media containing 5% FBS, 10 ng/mL bFGF, and 10 ng/mL EGF on gelatin (phase contrast, 10×)

FIG. 9 depicts a patient-derived ovarian cancer stem cell culture in serum-free media on gelatin (phase contrast, 10×)

FIG. 10 depicts a patient-derived ovarian cancer stem cell culture in serum-free media containing 10 ng/mL bFGF, and 10 ng/mL EGF on gelatin (phase contrast, 10×)

FIG. 11 depicts a small colony of patient-derived ovarian cancer stem cells in serum-free media containing 10 ng/mL bFGF, 10 ng/mL EGF, and 5 ng/mL activin A (phase contrast, 40×). Small cells with large nuclei that represents about 90% of the cell size can be observed in the compact colony suggesting very early cancer stem cells, (embryonic stem cell like, “early” OV-CSC).

FIG. 12A-D depicts a patient-derived ovarian cancer stem cell culture labeled for ovarian cancer biomarkers CA125 and MUC-1 (FIG. 12A). The cells were plated in a serum-free media containing 10 ng/mL bFGF and 10 ng/mL EGF. FIG. 12B depicts a red channel image of the cells of FIG. 12A that shows more than 90% of the cells are positive for CA125. FIG. 12C depicts a green channel image of the cells of FIG. 12A indicating that the majority of cells are positive for MUC-1 at various intensities. FIG. 12D depicts a blue channel image for bisbenzimide nuclear staining.

FIG. 13A-D depicts a patient-derived ovarian cancer stem cell culture labeled for ovarian cancer biomarker CK8 and proliferation marker Ki67 (FIG. 13A). The cells were plated in a serum-free media containing 10 ng/mL bFGF and 10 ng/mL EGF. FIG. 13B depicts a red channel image of the cells of FIG. 13A demonstrating intense proliferation (positive for Ki67 marker). FIG. 13C depicts a green channel image of the cells of FIG. 13A that shows the majority of the cells are positive for CK8. FIG. 13D depicts a blue channel image for bisbenzimide nuclear staining.

FIG. 14A-D depicts a patient-derived ovarian cancer stem cell culture labeled for cancer stem cell biomarkers EpCAM and NCAM. The cells were plated in a serum-free media containing 10 ng/mL bFGF and 10 ng/mL EGF. FIG. 14B depicts a red channel image of the cells of FIG. 14A that shows faint peri-nuclear expression of NCAM in some of the cells. FIG. 14C depicts a green channel image of the cells of FIG. 14A that depicts that the majority of the cells are positive for EpCAM and demonstrating the epithelial nature of the cells. FIG. 14D depicts a blue channel image for bisbenzimide nuclear staining.

FIG. 15A-D depicts a patient-derived ovarian cancer stem cell culture labeled for cancer stem cell biomarkers CD44 and nestin (FIG. 15A). The cells were plated in a serum-free media containing 10 ng/mL bFGF and 10 ng/mL EGF. FIG. 15C depicts a green channel image of the cells of FIG. 15A indicating that the majority of the cells are positive for CD44. FIG. 15B depicts a red channel image of the cells of FIG. 15A indicating some positive cells for nestin, mostly peri-nuclear. FIG. 15D depicts a blue channel image for bisbenzimide nuclear staining.

FIG. 16A-C depicts a patient-derived ovarian cancer stem cell culture labeled for the cancer stem cell biomarker CD133 (FIG. 16A). The cells were plated in a serum-free media containing 10 ng/mL bFGF and 10 ng/mL EGF. FIG. 16B depicts a green channel image of the cells of FIG. 16A indicating that the majority of the cells are positive for CD133. FIG. 16C depicts a blue channel image for bisbenzimide nuclear staining.

### DETAILED DESCRIPTION OF THE DISCLOSURE

The present disclosure provides a cell population obtained from human ovarian carcinoma (OV) tumors that consist mainly of high purity cancer stem cells. In embodiments, the purity of the cell population is at least 50%, at least 60%, at least 70%, at least 80%, at least 90%, at least 95%, or at least 99% cancer stem cells. These cancer stem cells are ovarian carcinoma progenitors and have the capacity of continuous self-renewal and differentiation to a certain level. The disclosure also concerns a method to produce a purified population of OV-derived stem cells, for further use as an antigen source for autologous immune therapy of cancer.

Testing and screening embodiments are also encompassed. The present disclosure uses the high purity OV stem cell population for genetic analysis to identify unique changes that drive the formulation of personalized medicines. The present disclosure provides a novel cell line that is modified in vitro, where this modification enhances the immune stimulatory characteristics of the OV. The OV cell line is an improvement over similar technologies using crude tumor preparations, as it provides a superior antigenic signal to noise ratio. The cell line lacks contaminant cell populations, such as fibroblasts, that could alter or diminish the in vitro or in vivo applications. The exemplary cell line of the present disclosure is also used for manufacturing of a drug for treating OV.

As used herein, the term “derived from,” in the context of peptides derived from one or more cancer cells, encompasses any method of obtaining the peptides from a cancer cell or a population of cancer cells. The cancer cell can be broken, for example, by a homogenizer or by osmotic bursting, resulting in a crude extract. Peptides, oligopeptides, and polypeptides of the crude extract can be exposed to dendritic cells, followed by processing of the peptides by the dendritic cells. The term “derived from” also encompasses intact cancer cells, where the cancer cells are living, or where the cancer cells have been treated with irradiation but are still metabolically active, or where the cancer cells have been treated with a cross-linking agent and therefore still comprise the peptides. “Derived from” also includes mixtures of cancer cell debris, free cancer cell proteins, and irradiated cancer cells, that therefore are derived from the cancer cells. “Derived from” also includes isolation or amplification of messenger RNA from the cancer cells, or cancer stem cells, for use in transfecting dendritic cells ex vivo to enable antigen presentation.

“Administration” as it applies to a human, mammal, mammalian subject, animal, veterinary subject, placebo subject, research subject, experimental subject, cell, tissue, organ, or biological fluid, refers without limitation to contact of an exogenous ligand, reagent, placebo, small molecule, pharmaceutical agent, therapeutic agent, diagnostic agent, or composition to the subject, cell, tissue, organ, or biological fluid, and the like. “Administration” can refer, e.g., to therapeutic, pharmacokinetic, diagnostic, research, placebo, and experimental methods. Administration can refer to in vivo treatment of a human or animal subject. Treatment of a cell encompasses contact of a reagent with the cell, as well as contact of a reagent with a fluid, where the fluid is in contact with the cell. “Administration” also encompasses in vitro and ex vivo treatments, e.g., of a cell, by a reagent, diagnostic, binding composition, or by another cell.

“Effective amount” encompasses, without limitation, an amount that can ameliorate, reverse, mitigate, prevent, or diagnose at least one symptom or sign of a medical condition or disorder. Unless dictated otherwise, explicitly or by context, an “effective amount” is not limited to a minimal amount sufficient to achieve a desired outcome nor limited to the optimal amount sufficient to achieve the desired outcome.

The severity of a disease or disorder, as well as the ability of a treatment to prevent, treat, or mitigate, the disease or disorder (achieve the desired outcome) can be measured, without implying any limitation, by a biomarker or by a clinical parameter. Biomarkers include blood counts, metabolite levels in serum, urine, or cerebrospinal fluid, tumor cell counts, cancer stem cell counts, tumor levels. Tumor levels can be determined by the Response Evaluation Criteria In Solid Tumors (RECIST) criteria (Eisenhauer, et al. (2009) Eur. J. Cancer. 45:228-247). Expression markers encompass genetic expression of mRNA or gene amplification, expression of an antigen, and expression of a polypeptide. Clinical parameters include progression-free survival (PFS), 6-month PFS, disease-free survival (DFS), time to progression (TTP), time to distant metastasis (TDM), and overall survival, without implying any limitation.

A composition that is “labeled” is detectable, either directly or indirectly, by spectroscopic, photochemical, biochemical, immunochemical, isotopic, or chemical methods. For example, useful labels include ³²P, ³³P, ³⁵S, ¹⁴C, ³H, ¹²⁵I, stable isotopes, epitope tags fluorescent dyes, electron-dense reagents, substrates, or enzymes, e.g., as used in enzyme-linked immunoassays, or fluorettes (disclosed in U.S. Pat. No. 6,747,135 which is incorporated by reference herein for all it discloses regarding fluorettes).

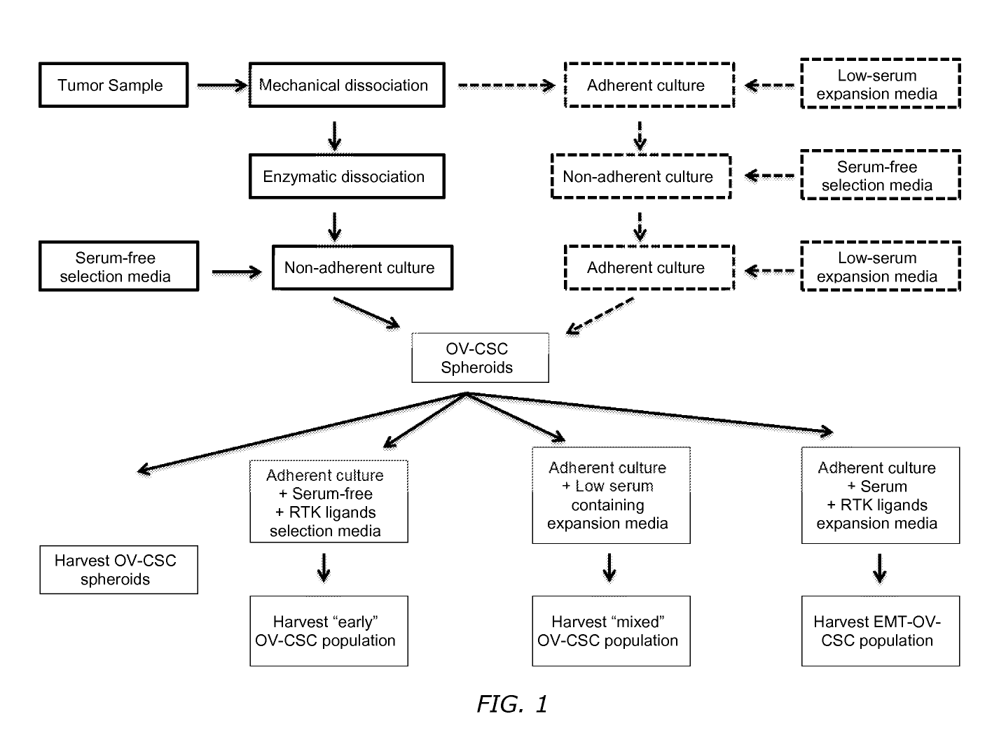

Therefore, disclosed herein are methods for preparing a population of purified spheroids, or single cells preparations derived from spheroids, of cancer stem cells, the method comprising acquiring a biopsy of OV, dissociating the cells of the biopsy, in vitro culturing the dissociated cells in a defined medium on a substrate, wherein the defined medium is supplemented with at least one growth factor that acts through the mitogen activated protein kinase (MAPK) pathway to yield a population of purified spheroids, or single cell preparations of OV stem cells. At least about 50%, at least about 60%, at least about 70%, or at least about 80% of the cancer stem cells in the population of purified OV-CSC express one or more of the biomarkers ATP-binding cassette sub-family G member 2 (ABCG2; GenBank Accession Number AAG52982.1), CD133, CD24, CD44, CD46, CD117, cytokeratin 18 (CK18), cytokeratin 8 (CK8), alpha fetoprotein (AFP), epithelial cell adhesion molecule (EpCAM; GenBank Accession Number NP_{—}002345.2), Ki-67, Nanog (GenBank Accession Number NM_{—}024865.2, NP_{—}079141.20), N-cadherin, neural cell adhesion molecule (NCAM; CD56), Oct3/4 (GenBank Accession Number NP_{—}002692.2; NP_{—}976034.4; NP_{—}001167002.1; NP_{—}068812.10), Slug (SNAI2)/Snail (SNAI1) (Slug/Snail), Twist, vimentin, cancer antigen-125 (CA-125), mucin 1 cell surface associated (MUC-1), human epididymis protein (He-4), aldehyde dehydrogenase (ALDH), cancer antigen 19-9 (CA19-9), human epidermal growth factor receptor 2 (HER2/neu), ganglioside CD2, estrogen receptor alpha, testosterone, transforming growth factor beta receptor (TGFβR), Sox2, epidermal growth factor receptor (EGFR), tumor-associated glycoprotein 72 (TAG-72), nestin, and tumor protein p53 (TP53). The OV-CSC do not substantially express any of carcinoembyronic antigen (CEA), follicle stimulating hormone (FSH), alpha human chorionic gonadotropin (αHCG), beta human chorionic gonadotropin (βHCG), and desmin. As used herein, the term “substantially” refers to cells, or populations of cells, in which the indicated markers are expressed on less than 20% of the cells. In other embodiments, the biomarkers are expressed on less than 15% of the cells, less that 10% of the cells, or less than 5% of the cells. A flow chart of the formation of the disclosed cell populations is presented in FIG. 1.

As used herein, the term “spheroids” refers to spherical aggregates of cancer stem cells formed by culture of cancer cells in serum-free medium. The ability to form spheroids is a characteristic of cancer stem cells.

In certain embodiments, at least about 50%, at least about 60%, at least about 70%, or at least about 80% of the cells in the OV-CSC spheroid population express two or more of the biomarkers EpCAM, CA-125, MUC-1, CD117, He-4, ALDH, CD133, CD24, Ki-67, CA19-9, HER2/neu, NCAM, ganglioside CD2, estrogen receptor alpha, vimentin, CK8, CK18, AFP, testosterone, TGFβR, EGFR, TAG-72, CD46, CD44, ABCG2, Slug/Snail, nestin, and TP53. In other embodiments, at least about 80% of the cells in the OV-CSC spheroid population express two or more of the biomarkers AFP, CK7, CK19, EpCAM, E-cadherin, Ov1, and OV6. In another embodiment, at least about 90% of the cells in the OV-CSC spheroid population express two or more of the biomarkers EpCAM, CA-125, MUC-1, CD117, He-4, ALDH, CD133, CD24, and Ki-67.

The spheroid population can be further expanded into one of three different subpopulations by altering culture conditions such as media composition and substrate. The characteristics of the bulk tumor, spheroid, early, mixed, and EMT OV-CSC populations are presented in Table 1.

TABLE 1
Summary of the conditions used to produce OV cell populations from bulk OV tumors

| | | | Markers | Conditions | Usefulness |
|---|---|---|---|---|---|
| Population | Availability | Cell type | (partial list) | for isolation | in therapy |
| Excised tumor, | immediate | OV* cells, | Mixed markers | Lysate | Diluted |
| bulk | | normal cells, | from any of the | and/or | antigenicity |
| | | very few OV- | cell populations | enzyme- | |
| | | CSC** | described below | dissociated | |
| Spheroids | 7-14 days | OV-CSC | At least two of | Non- | Proper |
| | | | EpCAM, CA-125, | adherent | antigenic |
| | | | MUC-1, CD117, | culture, | signal |
| | | | He-4, ALDH, | bFGF, | |
| | | | CD133, CD24, | EGF, | |
| | | | Ki-67 | Selection | |
| | | | Optionally CA19- | media | |
| | | | 9, HER2/neu, | | |
| | | | NCAM, | | |
| | | | ganglioside CD2, | | |
| | | | estrogen receptor | | |
| | | | alpha, vimentin, | | |
| | | | CK8, CK18, AFP, | | |
| | | | testosterone, | | |
| | | | TGFβR, EGFR, | | |
| | | | TAG-72, CD46, | | |
| | | | CD44, ABCG2, | | |
| | | | Snail/Slug, | | |
| | | | nestin, TP53 | | |
| | | | Does not express | | |
| | | | CEA, FSH, | | |
| | | | αHCG, βHCG, | | |
| | | | desmin | | |
| Colonies with | 14 days or | OV-CSC (very | At least two of | Adherent | Proper |
| small cuboid | longer | early, | EpCAM, CD133, | culture, | antigenic |
| cells, | | embryonic- | CD44, Nanog, | Selection | signal |
| “early” | | like) | Sox 2, Oct 3/4, | media, | |
| population | | | CD117, Ki-67 | bFGF, | |
| | | | Optionally | Activin A | |
| | | | CA-125, MUC-1, | | |
| | | | TGFβR, CD24 | | |
| Epithelial | 14 days or | More or less | At least two of | Adherent | Proper |
| monolayer, | longer | differentiated | EpCAM, CA-125, | culture, | antigenic |
| cells with | | OV-CSC | MUC-1, CD117, | Low calcium | signal |
| various sizes, | | (mixed) | CK8, CK18, Ki-67 | expansion | |
| small cuboid to | | | Optionally CA19- | media | |
| giant cells | | | 9, HER2/neu, | Optional, | |
| | | | NCAM, | EGF | |
| | | | ganglioside CD2, | | |
| | | | estrogen receptor | | |
| | | | alpha, | | |
| | | | testosterone, | | |
| | | | TGFβR, EGFR, | | |
| | | | TAG-72, CD46, | | |
| | | | He-4, ALDH, | | |
| | | | CD133, CD44, | | |
| | | | ABCG2, Nestin, | | |
| | | | TP53 | | |
| Monolayer with | 14 days or | EMT-OV-CSC | At least two of | Adherent | Proper |
| spindle or | and longer | (mesenchymal- | NCAM, | culture, | antigenic |
| irregular | | like) | Slug/Snail, Twist, | bFGF, | signal |
| shaped cells | | | CD24 | Expansion | |
| “EMT” | | | Optionally, | media | |
| population*** | | | CA125, MUC-1, | | |
| | | | N-cadherin, | | |
| | | | CD44, vimentin, , | | |
| | | | CD133, Nanog, | | |
| | | | CD117 | | |

*OV = Ovarian cancer;
**CSC = cancer stem cell;
***EMT = epithelial to mesenchymal transition

Furthermore, any of the early OV-CSC, mixed OV-CSC, or EMT-OV-CSC populations can be obtained from OV-CSC spheroids, early OV-CSC, mixed OV-CSC, or EMT-OV-CSC by changing the media and conditions as disclosed in Table 1.

In one embodiment, the OV-CSC spheroids are further cultured on an adherent substrate in the presence of activin A, FGF, and a serum-free media (selection media) to yield colonies with small cells referred to herein as an “early” population of OV-CSC which have characteristics of embryonic stem cells, and at least about 50%, at least about 60%, at least about 70%, or at least about 80% of the cells in the early OV-CSC population express two or more of biomarkers EpCAM, CD133, CD44, Nanog, Sox2, Oct3/4, CD117, and Ki-67. In another embodiment, at least about 80% of the cells in the early OV-CSC population express two or more of biomarkers EpCAM, CD133, CD44, Nanog, Sox2, Oct3/4, CD117, Ki-67, CA-125, MUC-1, TGFβR, and CD24. In another embodiment, at least about 90% of the cells in the early OV-CSC population express two or more of biomarkers EpCAM, CD133, CD44, Nanog, Sox2, Oct3/4, CD117, and Ki-67.

In another embodiment, the OV-CSC spheroids are further cultured on an adherent substrate under low calcium conditions in a serum-containing (expansion media) to yield colonies mixed with a monolayer wherein the cells have heterogeneous morphologies. The culture medium optionally includes EGF. These cells are referred to herein as a “mixed” population of OV-CSC which have a mixed differentiation profile, and at least about 50%, at least about 60%, at least about 70%, or at least about 80% of the cells in the mixed OV-CSC population express two or more of biomarkers EpCAM, CA-125, MUC-1, CD117, CK8, CK18, and Ki-67. In another embodiment, at least about 80% of the cells in the mixed OV-CSC population express two or more of biomarkers EpCAM, CA-125, MUC-1, CD117, CK8, CK18, Ki-67, CA19-9, HER2/neu, NCAM, ganglioside CD2, estrogen receptor alpha, testosterone, TGFβR, EGFR, TAG-72, CD46, He-4, ALDH, CD133, CD44, ABCG2, nestin, and TP53. In another embodiment, at least about 90% of the cells in the mixed OV-CSC population express two or more of biomarkers EpCAM, CA-125, MUC-1, CD117, CK8, CK18, and Ki-67.

In yet another embodiment, the OV-CSC spheroids are further cultured on an adherent substrate in the presence of FGF and a serum-containing media (expansion media) to yield a monolayer of spindle- or irregularly-shaped cells referred to herein as mesenchymal-like OV-CSC or “EMT-OV-CSC” (epithelial to mesenchymal transitioned [EMT] cancer stem cells). In this population, the spheroids have undergone a process of EMT characterized by the loss of the expression of at least one, or all, of the epithelial biomarkers CK8, CK18, and EpCAM. As used herein, loss of the expression of a biomarker refers to undetectable expression or expression in 40% (or less) of the cells, expression in 30% (or less) of the cells, expression in 20% (or less) of the cells, or expression in 10% (or less) of the cells. Additionally, the EMT process is characterized by the increase in the expression of at least one, or all, of the mesenchymal biomarkers Slug/Snail, Twist, CD44, NCAM, N-cadherin, and vimentin to at least 30%, at least 40%, at least 50%, at least 60%, at least 70%, at least 80%, at least 90%, at least 95%, or at least 99% of the cells in the population expressing the biomarker(s) of interest.

In one embodiment, at least about 50%, at least about 60%, at least about 70%, or at least about 80% of the cells in the EMT-OV-CSC population express two or more of the biomarkers NCAM, Slug/Snail, CD24, and Twist. In yet another embodiment, at least about 80% of the cells in the EMT-OV-CSC population express two or more of the biomarkers NCAM, Slug/Snail, CD24, Twist, CA-125, MUC-1, N-cadherin, CD44, vimentin, CD33, Nanog, and CD117. In yet another embodiment, at least about 90% of the cells in the EMT-OV-CSC population express two or more of the biomarkers NCAM, Slug/Snail, CD24, and Twist.

In certain embodiments of the cell populations, the cells express one or more of the indicated biomarkers. In other embodiments, the cells express two or more, three or more, four or more, five or more, six or more, seven or more, eight or more, nine or more, or ten or more of the indicated biomarkers. In yet other embodiments, the cells express 1, 2, 3, 4, 5, 6, 7, 8, 9, 10, 11, 12, 13, 14, 15, 16, 17, 18, 19, 20, 21, 22, 23, 24, or 25 of the indicated biomarkers.

Expression of biomarkers by a single cell, by a population of cells, or by a population of cells located in a specific structure such as a monolayer or a spheroid, can be determined by measuring expression of the polypeptide form of the biomarker or the mRNA form of the biomarker. Polypeptide expression can be measured using a labeled antibody, while nucleic acid expression can be measured by hybridization techniques, are available to the skilled artisan. Biomarkers that are not polypeptides or nucleic acids, such as oligosaccharides or small molecule metabolites, can also be measured by methods available to the skilled artisan.

Also disclosed herein are methods to obtain pure populations of isolated OV stem cells from ovarian tumor samples of various sizes (1 mg to grams). The tumor samples can be fresh or frozen, are dissociated by mechanical and/or enzymatic treatment, or are cultivated directly with minimal mechanical fragmentation.

Also disclosed herein, a non-adherent substrate is any biocompatible material with anti-biofouling properties or a coating with anti-biofouling properties (reduces accumulation of cells on a wetted surface) applied to a common culture surface. The coating can be applied using coating agents such as amino-silanes. If there is a non-adherent or anti-biofouling substrate, this substrate can be used for about 0-25 days, such as 0-21 days, 5-20 days, 5-10 days, 10-20 days, or any time period between zero and 25 days.

In another embodiment of the method that uses an adherent substrate, the adherent substrate can be one that is rich in RGD (Arg-Gly-Asp) tripeptide motifs (e.g., collagen, gelatin, MATRIGEL®). An adherent substrate is a surface that is configured to adhere to, and to collect, anchorage dependent cells. Moreover, the substrate can be an adherent substrate that is configured to adhere to and to collect anchorage dependent cells that are fibroblasts. RGD peptides can also be grafted on polymeric backbones such as polystyrene, hyaluronan, poly-lactic acid, or combinations thereof. The backbone can further carry proteoglycans. The proteoglycans can carry growth factors such as fibroblast growth factor (FGF), epidermal growth factor (EGF), activin A or follistatin.

A non-adherent substrate can cause fast and efficient enrichment of the cultures with cancer stem cells. A non-adherent substrate may be used when a large enough sample is provided, for example a surgically excised tumor, so that purification of OV-CSC can begin immediately. If the sample is very small, such as needle aspirate or peritoneal lavage, and non-adherent culture is not feasible, an adherent culture may be used for initial expansion, followed by a purification step on a non-adherent substrate, then followed by another expansion under adherent conditions. The alternative processing method is illustrated in FIG. 1 (dashed lines and boxes) and in detail below.

In certain culture embodiments, a first period of culture is provided on an adherent substrate, followed by a second period of culture on a non-adherent substrate. Also provided is a first period of culture on a non-adherent substrate, followed by a second period of culture on an adherent substrate. Periods can be, for example, one half day, 1 day, 2 days, 3 days, 4 days, 5 days, 6 days, 7 days, 8 days, 9 days, 10 days, 11 days, 12 days, 13 days, 14 days, 15 days, and the like, or any range thereof, such as 2-4 days, or 8-10 days, and so on. Additionally, the cycle can repeat such as an adherent culture followed by a non-adherent culture followed by an adherent culture, etc. In another embodiment, the cycle can repeat such as a non-adherent culture followed by an adherent culture, followed by a non-adherent culture, etc.

In another embodiment, the defined medium is supplemented with at least one growth factor that acts through the mitogen activated protein kinase (MAPK) pathway. In one embodiment, the growth factor is one or both of FGF and EGF, or an analogue thereof. In one embodiment, the FGF is basic fibroblast growth factor (bFGF). In another embodiment, the defined medium is supplemented with activin A. In another embodiment, the defined medium is not supplemented with activin A. Also disclosed is a defined medium supplemented with an agonist of activin A, in amount effective to prevent spontaneous differentiation of OV stem cells. In other embodiments, the defined media is supplemented by one or all of bone morphogenic protein (BMP) 2, 4, or 7.

Also provided is a OV-CSC cell line that is unique to each patient obtained from the patient's primary ovarian tumor, that (a) carries stem cell characteristics of self-renewal and pluripotency and the ability to differentiate; and (b) that carries a unique genomic cancerous signature in the majority of the cells, such as more than 50%.

The present disclosure encompasses nucleic acids, gene products, polypeptides, and peptide fragments, where identity can be reasonably established by a trivial name alone. Also encompassed, are nucleic acids, gene products, polypeptides, and peptide fragments, based on a particular GenBank Accession No., where the nucleic acid, polypeptide, and the like, has at least 50% sequence identity, at least 60%, at least 70%, at least 80%, at least 90%, at least 95%, at least 98%, at least 99%, or 100% identity sequence identity, to that of the GenBank No. where the biochemical function, or physiological function are shared, at least in part, or alternatively, irrespective of function.

Provided is a method wherein an immune response to cancer in a subject is stimulated with one of the compositions disclosed herein. The immune response that is stimulated comprises one or more of CD4⁺ T cell response, CD8⁺ T cell response, and B cell response. In certain embodiments, the CD4⁺ T cell response, CD⁺ T cell response, or B cell response, can be measured by ELISPOT assays, by intracellular cytokine staining (ICS) assays, by tetramer assays, or by detecting antigen-specific antibody production, according to assays that are known by persons of ordinary skill in the art. The immune response can comprise a survival time such as a 2-year overall survival (OS), and where the 2-year overall survival is at least 60%. An immune response in a patient can also be assessed by endpoints that are used in oncology clinical trials, including objective response (RECIST criteria), overall survival, progression-free survival (PFS), disease-free survival, time to distant metastasis, 6-month PFS, 12-month PFS, and so on.

Also disclosed herein are dendritic cells stimulated ex vivo with the OV-CSC, or antigens derived therefrom, for use in therapy of ovarian carcinoma. Encompassed herein are immunogenic compositions, such as vaccine compositions, comprising dendritic cells loaded with (exposed to) the OV-CSC ex vivo. In certain embodiments, the dendritic cells and tumor cells are from the same human subject (autologous) although embodiments where the dendritic cells and OV cells are from different subjects are within the scope of the present disclosure.

Dendritic cells can be loaded with OV tumor cell antigens comprising whole cells, cell lysates, cell extracts, irradiated cells or any protein derivative of an OV tumor cell, such as a OV-CSC. Dendritic cell immunogenic compositions can be prepared, and administered to a human subject by one or more routes of administration as are known to persons of ordinary skill in the art.

In certain embodiments, the OV-CSC cells are irradiated, or otherwise treated to prevent cell division, prior to loading with the dendritic cells. Alternatives to radiation include nucleic acid cross-linking agents that prevent cell division. Also provided is a method that uses of the OV-CSC population, as disclosed above, as a source of antigen for autologous immune therapy, for example, where the OV-CSC are inactivated by a radiant energy (e.g., gamma, UV, X), temperature (e.g., heat or cold), or chemical (e.g., cytostatic, aldehyde, alcohol) methods, or combinations thereof. In other embodiments, the OV-CSC are used as a source of antigen for ex vivo activation of dendritic cells.

The present disclosure provides prepared OV cells (OV-CSC), provides DC loaded with the prepared OV cells, and provides immunogenic compositions (or vaccines) comprising dendritic cells loaded the prepared OV cells. Without implying any limitation, an immunogenic composition of the present disclosure can comprise DC loaded with OV spheroids, loaded with a population of cells that comprises spheroids, loaded with a population of cells that was derived from spheroids and that were expanded on an adherent surface prior to loading on DC, loaded with spheroids that were subjected to homogenization or sonication prior to loading on DC, loaded with a population of expanded cells that were subjected to homogenization or sonication prior to loading on DC, and so on. In other embodiments, the DC are loaded with early OV-CSC, mixed OV-CSC, or EMT-OV-CSC.

Also disclosed herein is a population of OV-CSC that is capable of stimulating an effective immune response against a cell expressing at least one OV-specific antigen, wherein the OV-CSC population is contacted with at least one dendritic cell, wherein the OV-CSC population is processed in vivo or ex vivo by the dendritic cell, and wherein an effective immune response occurs in the subject in response to administration of the at least one dendritic cell to a subject.

An immune stimulatory amount of the disclosed compositions is, without limitation, an amount that increases ELISPOT assay results by a measurable amount, that increases ICS assay results by a measurable amount, that increases tetramer assay results by a measurable amount, that increases the blood population of antigen-specific CD4+ T cells by a measurable amount, that increases the blood population of antigen-specific CD8+ T cells by a measurable amount, or where the increase is by at least 10%, 20%, 30%, 40%, 50%, 60%, 70%, 80%, 90%, 100%, 1.5-fold, 2.0-fold, 3.0-fold, and the like, when compared to a suitable control. A suitable control can be a control composition, where dendritic cells are not loaded with OV cells, or are not loaded with peptide derived from OV cells.

The disclosure also provides pharmaceuticals, reagents, kits including diagnostic kits, that wherein the pharmaceuticals, reagents, and kits, comprise dendritic cells (DC), antibodies, or antigens. Also provided are methods for administering compositions that comprise at least one dendritic cell and at least one antigen, methods for stimulating antibody formation, methods for stimulating antibody-dependent cytotoxicity (ADCC), methods for stimulating complement-dependent cytotoxicity, and methods and kits for determining patient suitability, for determining patient inclusion/exclusion criteria in the context of a clinical trial or ordinary medical treatment, and for predicting response to the pharmaceutical or reagent. The pharmaceutical compositions, reagents, and related methods, of the disclosure encompass CD83 positive dendritic cells, where CD83 is induced by loading with IFN-gamma-treated, or untreated, cancer cells. In a CD83 aspect of the disclosure, the CD83 is induced by at least 2%, at least 3%, at least 4%, 6%, 7%, 8%, 9%, 10%, and the like. In another aspect, excluded are DC reagents, or DC-related methods, where CD83 on dendritic cells is not detectably induced by loading with IFN-gamma.

In one embodiment, a kit is provided which includes all of the reagents for generating OV-CSC spheroids, early OV-CSC, mixed OV-CSC, and/or EMT-OV-CSC from tumor samples according to the methods disclosed herein and/or reagents for characterizing the OV-CSC spheroids, early OV-CSC, mixed OV-CSC, and/or EMT-OV-CSC, and instructions for generating and/or characterizing OV-CSC spheroids, early OV-CSC, mixed OV-CSC, and/or EMT-OV-CSC. In another embodiment, the kit additionally, or alternatively, includes reagents and instructions for isolating dendritic cells, for loading the dendritic cells with OV-CSC, and/or for administering the DC-OV composition to a subject.

Tumor Sample Processing

The ovarian carcinoma (OV) stem cell population of the present disclosure can originate from fresh or frozen samples of patient tumor. The tumor sample can be a biopsy or a lavage of the peritoneal cavity containing OV cells. OV stem cells are isolated from needle biopsies and from the lavage fluid.

The tumor sample may be transported in a generic buffered media with a pH of about 7.4 (+/−0.6) such as RPMI, DMEM, F12, Williams, or combinations containing a protein source such as animal or human serum in concentrations from 0 to 100% or albumin at concentrations from 0 to 0.5% or macromolecules that ensure a physiological osmotic pressure. Examples of natural or artificial macromolecules are, but not limited to, hyaluronan, dextrans, polyvinyl alcohol. An antibiotic such as penicillin, streptomycin, gentramicyn in an optional combination with an antifungal such as amphotericin B, FUNGIZONE® (Life Technologies, Carlsbad, Calif.), can be used in the media to provide antimicrobial properties and reduce the risk of contamination during transportation.

The tumor sample can be kept below a metabolic active state by reducing the media temperature to 2 to 30° C., thus allowing the viability maintenance for a limited time (between 0 to 72 hours) before processing. Packaging (e.g., insulated packaging) may be used to ensure the proper temperature control during transportation.

The solid tumor tissue is then processed by mechanical dissociation using a sharp blade or tissue grinder device into small, less than 1 mm (on any dimension) fragments.

The solid tissue is optionally further processed by enzymatic dissociation. A variety of enzymes can be used to isolate single cells. Nonspecific proteolytic enzymes such as trypsin and pepsin can be used successfully. Targeting minimal cell membrane damage specific enzymes, including collagenase, dispase, elastase, hyaluronidase, or combinations thereof, may be used in the disclosed methods. Deoxyribonuclease (DNAse) can be used to degrade the free DNA from cell detritus responsible for unwanted stickiness of the cell preparation. After dissociation, the cells in suspension are washed from the excess enzyme and debris by straining through a 50-100 μm mesh and repeated centrifugation in a buffered saline (PBS, HBSS) or cell culture media.

Cell Culture Conditions and Spheroid Production

The single cell suspension described above is transferred in culture conditions that promote isolation, expansion of the stem cells and suppression of the differentiated and/or normal cells. This is accomplished by the congruence of the physical conditions, chemical environment, and manipulations.

The cell suspension is exposed to a non-adherent (anti-biofouling) substrate that does not allow cell attachment. Mature cells are commonly anchorage dependent and are rapidly eliminated when a proper adherent substrate is not provided. An anti-biofouling substrate can employ commercial products such as ultralow adherent flasks (Corning, Corning, N.Y.), polymers with natural hydrophobic properties (polyvinyl, polyethylene, polypropylene, fluoro-polymers) or coating with natural carbohydrate polymers such as agar-agar, starch, and the like.

The cancer stem cells will aggregate and/or clonally expand in spheroid formations that contain high purity cancer stem cells. A culture of cancer stem cell aggregates is shown in FIG. 3 having easily identifiable spheroid structures of various sizes. The mature cells will remain isolated and non-adherent. A differential gravitational separation can be used to select the larger spheroids from single cells, by simply allowing a timed vertical sedimentation or a short time low force centrifugation (less than 100×G). The selection method described is designed to accomplish the following: (a) eliminate of anchorage dependent cells that are, in general, mature, normal cells; (b) promote the clonal expansion in small clumps or spheroids of the young, stem cells that are anchorage independent; (c) promote the local autocrine activity as a result of clonal expansion of the stem cells; and (d) eliminate the autocrine source of activin A that is secreted by normal fibroblasts or ovarian cells.

The ability of cells to form spheres results, in part, from cell-surface proteins called integrins. Homophilic integrins expressed on the cell's surface ensure that cells of the same type “stay together”. Spheres are formed directly from enzyme digest which is a single cell suspension at the very beginning of a culture, or can be formed from frozen sample or an existing attached culture at any time. The enzyme digest seeding result in this spherical formations that incorporate the cells with the specific surface properties.

Fibroblasts, for example, are not incorporated into spheroids and are removed from a culture during gravitational feeding. The media used lacks molecules that promote adhesion in order to prevent the non-specific agglomeration of the cells not having homophilic proprieties and to prevent the adhesion to the culture vessel surfaces. Such cell adhesion molecules (CAMs) are commonly found in the animal or human serum. Therefore a media composition which is serum-free is suitable for culture of non-adherent spheroids.

In the serum-free media culture, supplements to the media may include any hormones, nutrients, mineral, and vitamins that are required for supporting growth and maintenance, or other desired aspects of cell physiology and function. In some instance one can stimulate and sustain the stem cell proliferation with the addition or adjustment of amount of growth factors that possess a mitogenic activity, such as the FGF family and EGF.

Spheres of cells (spheroids), including spheres of cancer stem cells, can be characterized in terms of biomarker expression by way of fixing and staining with labeled antibodies, followed by viewing with confocal microscopy. Biomarkers may also be measured by other immunochemistry methods, e.g., flow cytometry. Spheres can be prepared, for example, from suspensions obtained from fresh tumors, or from cells adapted to grow as adherent cells. The morphology of spheres, for example, large and irregular versus tiny and compact, may be influenced by the choice of medium.

In another embodiment, a cell population adherent to the anti-biofouling coating can be isolated based on aberrant activation of sonic hedgehog signaling mediated by protein kinase B (AKT) and focal adhesion kinase (FAK) signaling. These phenomena can be enhanced by modifications of the membranes induced by enzymes such as metalloproteases or enzymes used in dissociation (trypsin/collagenase). Such cell population can be associated with rapid proliferative and invasive tumors. Methods for assessing normal or aberrant activation of the sonic hedgehog signaling are available and known to persons of ordinary skill in the art.

Medium Used in Cell Culture

The defined media that is used to isolate the OV stem cells promotes cell survival and is specifically formulated for selection. The media is rich in carbohydrates and lipids but has minimal amount of protein (0.1%-3% albumin or 1%-5% serum). It contains not more than 1.5 mMol total calcium, does not contain inorganic iron compounds; rather, iron is completely bound to a transporter such as transferrin. The media is provided with an excess of essential and non-essential amino acids and essential lipids (alpha-linolenic and linoleic acids) (Table 4). Optionally, the media does not contain activin A and may contain an activin A receptor blocker such as follistatin. Also optionally, the media does not contain antioxidants such as superoxide dismutase (SOD) or catalase, but contains thiolic antioxidants such as glutathione.

In certain embodiments, the culture medium contains a low level of calcium, 0.1-1.5 mM, for example 10-150 mg/L in the form of calcium chloride.

The culture media consists in a basal formulation such as DMEM, F12, Williams, RPMI, Lebovitz supplemented with proteins (in certain formulations), amino acids, antioxidants, energetic substrate (glucose, galactose, L-glutamine), vitamins (B12), hormones (thyroid hormones, insulin) and growth factors (FGF, EGF) as depicted in Table 2.

The protein can be albumin in concentration of 0.1-0.5%, fetal bovine serum (FBS) 0.5%-20%. The protein can be substituted with macromolecules such as dextrans, hyaluronan, poly-vinyl alcohol in concentration ranging from 0.1% to 0.5%. The composition of such media is listed in Table 2, Table 3, and Table 4. The supplements are added into the media and mixed for feeding the cell cultures.

The media can be replaced in a three day a week schedule (e.g., Monday-Wednesday-Friday), or more frequently, e.g., every other day or daily, if the expansion is fast. A continuous feed or a micro-batch feed bioreactor can be used in the expansion phase.

The media contains growth factors that act through the MAPK pathway such as FGF and EGF. The concentration of these growth factors can vary between 0.1 to 100 ng/mL, commonly around 10 ng/mL.

TABLE 2
Basal media composition options for cancer stem cells:

| | M.W. | DMEM/F12 (1:1) | | William's E | | DMEM | | RPMI | | F12 | |
|---|---|---|---|---|---|---|---|---|---|---|---|
| Components | g/mole | mg/L | mM | mg/L | mM | mg/L | mM | mg/L | mM | mg/L | mM |

Amino Acids

| | | | | | | | | | | | |
|---|---|---|---|---|---|---|---|---|---|---|---|
| L-Alanine | 89.10 | 4.45 | 0.05 | 90 | 1.010 | | | | | 8.9 | 0.100 |
| L-Arginine | 174.20 | | | 50 | 0.287 | | | | | | |
| L-Arginine•HCl | 210.65 | 147.5 | 0.70 | | | 84 | 0.399 | 200 | 0.949 | 211 | 1.002 |
| L-Aspara- | 150.10 | 7.50 | 0.05 | 20 | 0.133 | | | 50 | 0.333 | 15.01 | 0.100 |
| gine•H₂O | | | | | | | | | | | |
| L-Aspartic Acid | 133.10 | 6.65 | 0.05 | 30 | 0.225 | | | 20 | 0.150 | 13.3 | 0.100 |
| L-Cysteine | 121.16 | | | 40 | 0.330 | | | | | | |
| L-Cyste- | 175.65 | 17.56 | 0.10 | | | | | | 0.000 | 35.12 | 0.200 |
| ine•HCl•H₂O | | | | | | | | | | | |
| L-Cystine•2HCl | 313.11 | 31.29 | 0.10 | 26.07 | 0.083 | 62.57 | 0.200 | 65.15 | 0.208 | | |
| L-Glutamic Acid | 147.10 | 7.35 | 0.05 | 50 | 0.340 | | | 20 | 0.136 | 14.7 | 0.100 |
| L-Glutamine | 146.10 | 365 | 2.50 | 292 | 1.999 | 584 | 3.997 | 300 | 2.053 | 146 | 0.999 |
| L-Glycine | 75.10 | 18.75 | 0.25 | 50 | 0.666 | 30 | 0.399 | 10 | 0.133 | 7.5 | 0.100 |
| L-Histidine | 155.16 | | | 15 | 0.097 | | | | | | |
| L-Histi- | 209.65 | 31.48 | 0.15 | | | 42 | 0.200 | 15 | 0.072 | 20.96 | 0.100 |
| dine•HCl•H₂O | | | | | | | | | | | |
| L-Hydroxyproline | 131.13 | | | | | | | 20 | 0.153 | | |
| L-Isoleucine | 131.20 | 54.47 | 0.42 | 50 | 0.381 | 105 | 0.800 | 50 | 0.381 | 3.94 | 0.030 |
| L-Leucine | 131.20 | 59.05 | 0.45 | 75 | 0.572 | 105 | 0.800 | 50 | 0.381 | 13.1 | 0.100 |
| L-Lysine•HCl | 182.65 | 91.25 | 0.50 | 87.46 | 0.479 | 146 | 0.799 | 40 | 0.219 | 36.5 | 0.200 |
| L-Methionine | 149.20 | 17.24 | 0.12 | 15 | 0.101 | 30 | 0.201 | 15 | 0.101 | 4.48 | 0.030 |
| L-Phenylalanine | 165.20 | 35.48 | 0.21 | 25 | 0.151 | 66 | 0.400 | 15 | 0.091 | 4.96 | 0.030 |
| L-Proline | 115.10 | 17.25 | 0.15 | 30 | 0.261 | | | 20 | 0.174 | 34.5 | 0.300 |
| L-Serine | 105.10 | 26.25 | 0.25 | 10 | 0.095 | 42 | 0.400 | 30 | 0.285 | 10.5 | 0.100 |
| L-Threonine | 119.10 | 53.45 | 0.45 | 40 | 0.336 | 95 | 0.798 | 20 | 0.168 | 11.9 | 0.100 |
| L-Tryptophan | 204.20 | 9.02 | 0.04 | 10 | 0.049 | 16 | 0.078 | 5 | 0.024 | 2.04 | 0.010 |
| L-Tyro- | 261.20 | 55.79 | 0.21 | 50.65 | 0.194 | 103.8 | 0.397 | 28.83 | 0.110 | 7.8 | 0.030 |
| sine•2Na•2H₂O | | | | | | | | | | | |
| L-Valine | 117.10 | 52.85 | 0.45 | 50 | 0.427 | 94 | 0.803 | 20 | 0.171 | 11.7 | 0.100 |

Sugar

| | | | | | | | | | | | |
|---|---|---|---|---|---|---|---|---|---|---|---|
| D-Glucose | 180.00 | 3151 | 17.51 | 2000 | 11.111 | 4500 | 25 | 2000 | 11.11 | 1802.00 | 10.01 |

Vitamins/Nucleotides/Minute Organics

| | | | | | | | | | | | |
|---|---|---|---|---|---|---|---|---|---|---|---|
| Ascorbic acid | 176.13 | | | 2 | 1.14E−02 | | | | | | |
| Vitamin B-12 | 1355 | 0.6800 | 5.02E−04 | 0.2 | 1.48E−04 | | | 0.005 | 3.69E−06 | 1.4 | |
| (cobalamin) | | | | | | | | | | | |
| Biotin | 244.00 | 0.0035 | 1.43E−05 | 0.5 | 2.05E−03 | | | 0.2 | 8.20E−04 | 0.0073 | 2.99E−05 |
| Choline | 140.00 | 8.98 | 6.41E−02 | 1.5 | 1.07E−02 | 4 | 2.86E−02 | 3 | 2.14E−02 | 13.96 | 0.099714 |
| chloride | | | | | | | | | | | |
| Ergocalciferol | 396.66 | | | 0.1 | 2.52E−04 | | | | | | |
| Folic acid | 441.00 | 2.65 | 6.01E−03 | 1 | 2.27E−03 | 4 | 9.07E−03 | 1 | 2.27E−03 | 1.3 | 2.95E−03 |
| I-inositol | 180.00 | 12.60 | 7.00E−02 | 2 | 1.11E−02 | 7.2 | 4.00E−02 | 35 | 1.94E−01 | 18 | 0.1 |
| Menadione | | | | 0.01 | | | | | | | |
| sodium | | | | | | | | | | | |
| bisulfate | | | | | | | | | | | |
| Niacinamide | 122.00 | 2.02 | 1.66E−02 | 1 | 8.20E−03 | 4 | 3.28E−02 | 1 | 8.20E−03 | 0.037 | 3.03E−04 |
| D-Calcium | 477.00 | 2.21 | 4.63E−03 | 1 | 2.10E−03 | 4 | 8.39E−03 | 0.25 | 5.24E−04 | 0.48 | 1.01E−03 |
| pantothenate | | | | | | | | | | | |
| Pyridoxal HCl | 204.00 | 2.00 | 9.80E−03 | 1 | 4.90E−03 | 4 | 1.96E−02 | | | | |
| Pyridoxine HCl | 206.00 | 0.03 | 1.50E−04 | | | | | 1 | 4.85E−03 | 0.062 | 3.01E−04 |
| Riboflavin | 376.00 | 0.22 | 5.82E−04 | 0.1 | 2.66E−04 | 0.4 | 1.06E−03 | 0.2 | 5.32E−04 | 0.038 | 1.01E−04 |
| Thiamine HCl | 337.00 | 2.17 | 6.44E−03 | 1 | 2.97E−03 | 4 | 1.19E−02 | 1 | 2.97E−03 | 0.34 | 1.01E−03 |
| (Vitamin B1) | | | | | | | | | | | |
| Thymidine | 242.23 | 0.37 | 2.07E−03 | | | | | | | 0.7 | |
| Putres- | 88.15 | 0.08 | 9.19E−04 | | | | | | | 0.161 | |
| cine•2HCl | | | | | | | | | | | |
| Sodium pyruvate | 110.00 | 55.00 | 5.00E−01 | 25 | 0.227 | | | | | 110 | |
| a-Tocopherol | | | | 0.01 | | | | | | | |
| phosphate | | | | | | | | | | | |
| Lipoic acid | 206.00 | 0.11 | 5.10E−04 | | | | | | | 0.21 | |
| Linoleic acid | 280.48 | 0.04 | 1.50E−04 | | | | | | | 0.08 | |
| Para- | | | | | | | | 1 | | | |
| aminobenzoic | | | | | | | | | | | |
| acid | | | | | | | | | | | |
| Vitamin A | | | | 0.1 | | | | | | | |
| acetate | | | | | | | | | | | |

Inorganic Bulk Salts, buffers

| | | | | | | | | | | | |
|---|---|---|---|---|---|---|---|---|---|---|---|
| Magnesium | 95.21 | 28.64 | 0.30 | | | | | | | 57.22 | 0.601 |
| chloride, | | | | | | | | | | | |
| anhydrous | | | | | | | | | | | |
| Magnesium | 120.40 | 48.84 | 0.41 | 97.67 | 0.81 | 97.67 | 0.8112 | 48.84 | 0.41 | | |
| sulfate, | | | | | | | | | | | |
| anhydrous | | | | | | | | | | | |
| Potassium | 74.55 | 311.80 | 4.18 | 400 | 5.37 | 400 | 5.3655 | 400 | 5.37 | 223.6 | 3.00 |
| chloride | | | | | | | | | | | |
| Sodium | 142.00 | 71.02 | 0.50 | | | | | 800 | 5.63 | | |
| phosophate, | | | | | | | | | | | |
| dibasic, | | | | | | | | | | | |
| anhydrous | | | | | | | | | | | |
| Sodium | 160.00 | | | | | 125 | 0.7813 | | | | |
| phosophate | | | | | | | | | | | |
| dibasic•H₂O | | | | | | | | | | | |
| Sodium | 58.44 | 6999.50 | 119.77 | 6800 | 116.36 | 6400 | 109.5140 | 6000 | 102.67 | 7599 | 130.03 |
| chloride | | | | | | | | | | | |
| Sodium | 120.00 | 62.50 | 0.52 | 140 | 1.17 | | | | | | |
| phosphate | | | | | | | | | | | |
| monobasic•H₂O | | | | | | | | | | | |
| Calcium | 111.00 | 116.60 | 1.05 | 200 | 1.80 | 200 | 1.8018 | | | 33.22 | 0.30 |
| chloride, | | | | | | | | | | | |
| anhydrous | | | | | | | | | | | |
| Calcium | 236.00 | | | | | | | 100 | 0.42 | | |
| nitrate•4H₂O | | | | | | | | | | | |
| Sodium | 84.01 | 2438.0 | 29.02 | 2200 | 26.19 | 3700 | 44.0424 | 2000 | 23.81 | 1176 | 14.00 |
| bicarbonate | | | | | | | | | | | |
| Hepes buffer | | | | | | | | | | 142.04 | |
| (1M) | | | | | | | | | | | |

Trace Minerals

| | | | | | | | | | |
|---|---|---|---|---|---|---|---|---|---|
| Cupric | 249.70 | 0.0013 | 5.21E−06 | 0.0001 | 4E−07 | | | 0.0025 | 1.00E−05 |
| sulfate•5H₂O | | | | | | | | | |
| Ferrous | 278.00 | 0.42 | 1.50E−03 | | | | | 0.834 | 0.003 |
| sulfate•7H₂O | | | | | | | | | |
| Ferric | 101.10 | 0.05 | 4.95E−04 | 0.0001 | 9.9E−07 | 0.1 | 0.0010 | | |
| nitrate•9H₂O | | | | | | | | | |
| Zinc | 287.50 | 0.43 | 1.50E−03 | | | | | | |
| sulfate | | | | | | | | | |
| Zinc | | | | 0.0002 | | | | 0.863 | |
| sulfate•7H₂O | | | | | | | | | |
| Manganese | | | | 0.0001 | | | | | |
| chloride•4H₂O | | | | | | | | | |

Others

| | | | | | |
|---|---|---|---|---|---|
| Na | 2.39 | | | | 4.77 |
| hypoxanthine | | | | | |
| Phenol red | 8.10 | 10 | 15 | 5 | 1.2 |
| Glutathione | | 0.05 | | 1 | |
| (reduced) | | | | | |
| Methyl | | 0.03 | | | |
| linoleate | | | | | |

TABLE 3
Lineage stem cell supplement (50 mL units for reconstitution in 1 L
of basal media)

| | Formulation | |
|---|---|---|
| | (per 50 mL supplement or | |
| | 1 L of final media) | |
| Components | value | unit |
| Water | QS to 50 | ml |
| human serum albumin | 2.5 | g |
| Transferrin partially saturated | 20 | mg |
| Insulin | 20 | mg |
| T3 | 0.002 | mg |
| Selenite | 0.01 | mg |
| Progesterone | 0.005 | mg |
| Putrescine | 10 | mg |
| Catalase | 2.5 | mg |
| Glutathione | 1 | mg |
| Carnitine | 2 | mg |
| Biotin | 0.05 | g |
| L-glutamine | 365 | mg |
| Ethanolamine | 15 | mg |
| HEPES | 1 | g |
| Lipid Mix (see Table 4) | 5 | ml |

TABLE 4
Lipid mix

| | Concentration: |
|---|---|
| Components | μg/mL |
| Linolenic | 10 |
| Linoleic | 10 |
| Tocopherol acetate | 50 |
| Cholesterol | 100 |

The lipid mix is made by o/w emulsions using Pluronic F68, phosphatidyl choline, Tween 80, cyclodextrin, or combinations thereof

In one embodiment, the media is supplemented with FGF at about 0.1 to 100 ng/mL, at about 0.5-50 ng/mL, at about 1-40 ng/mL, at about 2-30 ng/mL, at about 3-20 ng/mL, at about 5-15 ng/mL, at about 6-14 ng/mL, at about 7-13 ng/mL, at about 8-12 ng/mL, at about 9-11 ng/mL, or at about 10 ng/mL. In other embodiments FGF is present in the media at about 5 ng/mL, at about 6 ng/mL, at about 7 ng/mL, at about 8 ng/mL, at about 9 ng/mL, at about 11 ng/mL, at about 12 ng/mL, at about 12 ng/mL, at about 14 ng/mL, or at about 15 ng/mL.

In another embodiment, the media is supplemented with EGF at about 0.1 to 100 ng/mL, at about 0.5-50 ng/mL, at about 1-40 ng/mL, at about 2-30 ng/mL, at about 3-20 ng/mL, at about 5-15 ng/mL, at about 6-14 ng/mL, at about 7-13 ng/mL, at about 8-12 ng/mL, at about 9-11 ng/mL, or at about 10 ng/mL. In other embodiments EGF is present in the media at about 5 ng/mL, at about 6 ng/mL, at about 7 ng/mL, at about 8 ng/mL, at about 9 ng/mL, at about 11 ng/mL, at about 12 ng/mL, at about 12 ng/mL, at about 14 ng/mL, or at about 15 ng/mL.

Also provided is a medium which is not supplemented with one or both of superoxide dismutase (SOD) or catalase. The use of antioxidants can have both positive and negative consequences. Cancer stem cells are far more tolerant than normal cells to free radicals and glycolytic metabolism. Therefore in suboptimal cultures such as high density, infrequent media replacement, high concentration of metabolites in the media, it is most likely that the normal sensitive cells to be eliminated first. By not including antioxidants in the media, a population of cells can be selected that is likely to be of a cancerous origin, more resistant than the normal cells. Therefore, in certain embodiments, antioxidants, such as catalase and inhibitors of SOD are added to the culture medium and in other embodiments, these compounds are omitted from the culture media.

In an alternative method, the activin/follistatin system can be used to isolate very early cancer stem cells. The addition of activin A can select a subpopulation of activin A-resistant OV stem cells. Follistatin is used to block the activin A receptors and prevent spontaneous differentiation of the OV stem cells, especially when large numbers of cells that endogenously secrete activin A are present, such as fibroblasts and normal ovarian cells. The use of follistatin has no effect if the cells are insensitive to activin A or in high purity OV stem cell populations where follistatin can be secreted endogenously.

Activin A is a protein that is a member of the transforming growth factor-beta (TGF-beta) superfamily. When added or included in culture medium, activin helps maintain stem cell pluripotency and self-renewal. However, activin A promotes maturation and differentiation of young ovarian cells and cancer cells that are receptive. Therefore, an initial goal is in vitro fast expansion of the tumor that also sustains the proliferation of cancer stem cells by creating a proper autocrine environment in the culture. Although activin A may select a subpopulation of very young cancer stem cells, such conditions applied early in the manufacturing will greatly delay the expansion given the very low concentration of the hepatic cancer stem cells in the bulk. For example, a “fast expansion” is an expansion that results in the media in the culture vessels having obvious signs of consumption (change of pH for example) and the number of cells is visibly higher every day reflected by increased confluence.

For fast expansion, activin A is preferably omitted and not added, because it will slow down the culture growth. For some applications the interest is to obtain a very early stem cell population and the use of the activin A will select that cell population. Therefore, in one embodiment, an activin A-containing expansion is initiated and a first composition is administered to a subject comprising the activin A-activated cultured cells, followed by the isolation of the activin A-insensitive cells in an activin-A free culture and administering this second composition comprising the activin A free cultured cells to the subject.

In one embodiment, the media is supplemented with activin A at about 0.01 to 10 ng/mL, at about 0.05-9 ng/mL, at about 0.1-8 ng/mL, at about 0.5-7 ng/mL, at about 1-6 ng/mL, at about 1-5 ng/mL. In other embodiments, activin A is present in the media at about 0.5 ng/mL, at about 0.7 ng/mL, at about 0.9 ng/mL, at about 1 ng/mL, at about 1.25 ng/mL, at about 1.5 ng/mL, at about 1.75 ng/mL, at about 2 ng/mL, at about 2.25 ng/mL, at about 2.5 ng/mL, at about 2.75 ng/mL, at about 3 ng/mL, at about 3.5 ng/mL, at about 4 ng/mL, at about 4.5 ng/mL, at about 5 ng/mL, at about 6 ng/mL, at about 7 ng/mL, at about 8 ng/mL, at about 9 ng/mL, or at about 10 ng/mL.

Also disclosed is an embodiment wherein the media is supplemented with an antagonist of activin A, such as, but not limited to, follistatin or an antibody that specifically binds to activin A.

In another embodiment, the media is supplemented with follistatin at about 0.1 to 100 ng/mL, at about 0.5-50 ng/mL, at about 1-40 ng/mL, at about 2-30 ng/mL, at about 3-20 ng/mL, at about 5-15 ng/mL, at about 6-14 ng/mL, at about 7-13 ng/mL, at about 8-12 ng/mL, at about 9-11 ng/mL, or at about 10 ng/mL. In other embodiments, follistatin is present in the media at about 5 ng/mL, at about 6 ng/mL, at about 7 ng/mL, at about 8 ng/mL, at about 9 ng/mL, at about 11 ng/mL, at about 12 ng/mL, at about 12 ng/mL, at about 14 ng/mL, or at about 15 ng/mL.

The combination of mitogens (e.g., FGF/EGF), activin A, and adherent substrate may result in an increase in the proliferation of normal cells such as fibroblasts or stellate cells. Thus, conditions are created to promote the expansion of very early OV stem cells or progenitors that are insensitive to activin A in a rich environment or “stroma” constituted by cells with nourishing or encapsulating properties (e.g., fibroblasts, stellate cells). The colonies of OV are progressively observed to develop along and spatially displace the stroma in the course of the next few days to weeks of cell culture. The media used in this method is the combination of the formulations described in Tables 2, 3 and 4.

There is a relationship between FGF, EGF, and activin A, and “very early” OV cancer stem cells. FGF and EGF cause proliferation of OV stem cells in any differentiation status including the very early ones. Where activin A is in the cell culture medium, the activin A is permissive for (allows) proliferation exclusively of the very early OV stem cells that are insensitive to activin A. If the OV stem cells become sensitive, the proliferation will be stopped or reduced by activin A.

Insensitivity to FGF and EGF is not common and there are no natural blockers. Insensitivity to activin A can be mediated by follistatin, a natural blocker of the activin receptor. Follistatin can be secreted by the same tumor cell or by cells surrounding the tumor. Activin A is typically secreted by the cells surrounding the tumor, therefore it is possible that the expansion of the tumor is dependent on the surrounding cells (inhibiting) and by the tumor (promoting the expansion). The lack of receptor for activin A, a characteristic of the very early, undifferentiated cancer stem cells can prevent the control of the tumor by the surrounding tissue.

The in vitro cultures will contain embryonic stem cell-like colonies. These colonies may be surrounded by stromal cells, that can be normal fibroblasts, differentiated tumor cells, or mesenchymal transitioned tumor cells.

The present disclosure provides method for preparing OV-CSC where the total culturing time including time required for manipulations such as changing media, replating, centrifugation, and sedimenting, is less than five months, less than four months, less than three months, less than two months, less than one month, less than 150 days, less than 120 days, less than 90 days, less than 60 days, less than 30 days, or less than 150 days (+/−20 days), less than 120 days (+/−20 days), less than 90 days (+/−20 days), less than 60 days (+/−20 days), less than 30 days (+/−20 days). In exclusionary embodiments, the present disclosure can exclude any method for preparing cancer stem cells, and any population of cancer stem cells prepared by that method, where time required for manipulation is greater than one of the time-frames disclosed above. Also provided is a time in adherent culture that is indicated by one of the above time-frames. Also provided is a time in non-adherent culture that is one of the above time-frames. Moreover, provided is a combined time in adherent culture and in non-adherent culture that is identified by one of the above time-frames.

Epithelial to Mesenchymal Transition (EMT)

Tumors of epithelial origin are known to regress or trans-differentiate into a mesenchymal state. Epithelial phenotypes are immobile, contribute to volume growth of the tumor limited to the originating tissue and are typically more differentiated. When EMT occurs, the cells gain mobility and produce adjacent tissue infiltration and distant metastases. The transitioned cell also gains a stem cell-like phenotype, with the ability to replicate and differentiate resulting in a new tumor (metastasis) in the host tissue with characteristics of the originating (primary) tumor. By EMT, the tumor cells gain additionally immunosuppressive ability, drug pump and radioresistance.

The media composition and the physical selection method promote the EMT phenomenon in vitro. The advantage of using an EMT transitioned population as an immunogen is in prevention of tumor recurrences. The antigenicity of EMT cancer cells could enable the immune system to recognize and destroy mobile cancer cells that cause metastasis. In the process of metastasis these cells travel in very low number, seed the host tissue, revert to an epithelial phenotype (MTE transition), grow and form a new tumor that has similar characteristics with the primary tumor. The conditions necessary to cause in vitro EMT are spheroid formation in serum free media, stimulation with bFGF, stimulation with BMP2, 4, or 7, then plating on adherent substrate containing RGD (Arg-Gly-Asp) peptide motifs (e.g., collagen, gelatin, etc).

The EMT-OV-CSC subpopulation is obtained by culturing OV spheroids, or early OV or mixed OV, under culture conditions as described in Table 1 and FIG. 1.

As used herein, the term “OV-CSC” can generally refer to OV-CSC spheroids, early OV-CSC, mixed OV-CSC, or EMT-OV-CSC.

Obtaining OV-CSC from Small Sources of Tumor

An alternative method for OV stem cell selection is used when the number of sample cells is small. For exemplification, a small number of viable cells obtained from a tumor is less than 10×10⁶ viable cells after enzymatic dissociation. For the purposes of this disclosure a small sample refers to a sample obtain for example from a needle biopsy or peritoneal lavage, in contrast to a sample obtained from an excised tumor, which is typically not considered a small sample and weighs at least 0.5 to 5-10 grams. Core biopsies are done with 18 or 16 or 14 gauge needles, resulting in 5-50 mg samples. A relatively new procedure called a vacuum assisted biopsy is also done with an 11 gauge needle, and a vacuum assisted device (VAD). An 11 gauge probe paired with a vacuum-assisted device typically picks up 94 mg with each core sample. The 14 gauge needle with vacuum assistance typically picks up 37 mg, but only 17 mg when paired with an automated biopsy gun. In this alternative method, depicted in FIG. 1 (dashed arrows and boxes), cells obtained from the tumor sample are transferred, before or after dissociation, to an adherent substrate containing RGD (Arg-Gly-Asp) rich compounds (e.g., collagen, gelatin or MATRIGEL®) and in the presence of a selection (serum-free) culture media described herein. The selection method described is designed to (a) promote initial clonal expansion of the individual cancer stem cells that are present in low number, and (d) promote the local autocrine activity as a result of clonal expansion of the stem cells.

Adherent substrates are RGD rich proteins such as collagen or gelatin. The substrate can be constructed by attaching the protein or peptide to various materials such as polystyrene polycarbonate, cyclic olefin copolymer or glass. The RGD peptide can be grafted on polymeric backbones such as hyaluronic acid, polylactic acid and combinations. Such polymers can be further enhanced with carrier terminations for growth factors such as proteoglycans (e.g., heparin sulfate, chondroitin sulfate, keratin sulfate, and so on).

The cell culture surface can be used directly or using coating agents such as aminosilanes. A coating is a compound that has adherent property (substrate) for the cells and is applied on top of the growth vessel's material. It can be a natural compound such as collagen or gelatin and also can be constructed of a more synthetic polymer having the mentioned radicals/terminations. A coating agent (glue, such as silanes) can be used to improve the adherence of the coating to the culture vessel material (for example to glass). Silanes alone can be used if they contain the desired radicals or terminal groups.

With this method and formulation, a large number of cells can be obtained in relatively short period of time. Starting from a few milligrams, cultures of tissue samples, such as needle biopsies containing 10³ to 10⁶ cells, can be expanded in 3-4 weeks to about 10⁸ cells.

Expansion of OV Stem Cell Cultures and Generation of Subpopulations

The OV-CSC can be propagated and expanded indefinitely, as an additional characteristic of stem cells.

Furthermore, the OV-CSC can be partial or totally differentiated. If the stem cell expansion conditions are removed, the OV stem cells can slow down or stop the proliferation, and change morphology and phenotype to a more differentiated cell type. The morphology can become flat, epitheloid or stelate having multiple nuclei—a characteristic of the more mature ovarian cells or stelate cells.

The adherent cultures can be dissociated in single cell suspension and transferred to non-adherent (anti-biofouling) conditions to remove the anchorage dependent differentiated cells. After 2-3 days, the stem cells tend to aggregate and clonally expand in small spheroids that based on differential sedimentation can be separated from the single cells. The spheroids can be re-plated in adherent conditions and further propagated. This method will purify the culture stem cell content if the cultures are overtaken by differentiated cells or normal cells such as fibroblasts, from 1-30% to 90-99% stem cell content. The method can be repeated as many times needed in order to restore stem cell purity.

Small spheroids generally have the dimensions of between 0.1 mm and 2 mm. The size distribution, in terms of number of cells per small spheroid, is generally between 10 cells and 10,000 cells. The shape of a small spheroid can be spherical or oval, and can also occur as conglomerates of spherical or oval structures.

A patient-specific OV-CSC cell line can be used to identify the genomic mutation responsible for the neoplastic transformation when compared with normal tissue from the same patient. The genomic mutation may not be expressed in every stage of differentiation. Some regulatory proteins, or transcription factors, are only temporary expressed and may disappear during maturation, resulting in a malformed cell but with normal proteins. Identification of a cell population that is maximally expressing the mutation and exposing this population to the immune system could be a major advantage of using cancer stem cells as an antigen source for immune-therapy

By identifying the genomic mutation a personalized formulation can be created for a cancer treatment, for example a small molecule, a DNA sequence, antisense RNA or combinations.

Such cell lines can be further used to create screening plates (96 wells for example) for drug discovery. Multiple lines from various patients can be combined in a single plate to address variability between individuals.

Ovarian carcinoma cancer stem cells may retain some properties of the originating tissue such as secretion of proteins, growth factors and hormones (functional tumors). These properties can be exploited given the immortal characteristics of the cell lines, to produce “self” proteins that can be used for the same patients (for example albumin, transforming growth factor (TGF), insulin, glucagon, DOPA etc). The cells can be introduced in small bioreactors and the secretion product collected, purified and stored for the same patient use. This method is particularly advantageous that the patient will not develop immune resistance such as the more traditional biosimilars.

Loading Dendritic Cells

The individual OV-CSC cells obtained from the patient can be used to produce an antigen for immune therapy. The advantage of using the purified stem cell line resides in a better signal to noise ratio. The more mature cells from the tumor may have compensatory mechanisms that can mask the antigenicity and could be not identified by the immune system. As an antigen source, the OV-CSC can be used alive, mitotically inactive, nonviable or fragmented. Various methods can be used to modify the cells for optimal antigen exposure: a radiant energy (e.g., gamma, UV, X), temperature (e.g., heat or cold), or chemical (e.g., cytostatic, aldehyde, alcohol) or combinations.

In exemplary implementations, the present disclosure encompasses reagents and methods for activating dendritic cells (DCs), with one or more immune adjuvants, such as GM-CSF, a toll-like receptor (TLR) agonist, e.g., CpG-oligonucleotide (TLR9), imiquimod (TLR7), poly(I:C) (TLR3), glucopyranosyl lipid A (TLR4), murein (TLR2), flagellin (TLR5), as well as an adjuvant such as CD40 agonists, e.g., CD40-ligand, or the cytokine, interferon-gamma, prostaglandin E2, and the like. See, e.g., U.S. Pat. No. 7,993,659; U.S. Pat. No. 7,993,648; U.S. Pat. No. 7,935,804, each of which is incorporated herein by reference for all it discloses regarding activating DCs. The present disclosure encompasses ex vivo treatment of DCs with one or more of the above adjuvant reagents, or in addition, or alternatively, administration of the adjuvant to a human subject, animal subject, or veterinary subject.

The immune system encompasses cellular immunity, humoral immunity, and complement response. Cellular immunity includes a network of cells and events involving dendritic cells, CD8⁺ T cells (cytotoxic T cells; cytotoxic lymphocytes), and CD4⁺ T cells (helper T cells). Dendritic cells (DCs) acquire polypeptide antigens, where these antigens can be acquired from outside of the DC, or biosynthesized inside of the DC by an infecting organism. The DC processes the polypeptide, resulting in peptides of about ten amino acids in length, transfers the peptides to either MHC class I or MHC class II to form a complex, and shuttles the complex to the surface of the DC. When a DC bearing a MHC class I/peptide complex contacts a CD8⁺ T cell, the result is activation and proliferation of the CD8⁺ T cell. Regarding the role of MHC class II, when a DC bearing a MHC class II/peptide complex contacts a CD4⁺ T cell, the outcome is activation and proliferation of the CD4⁺ T cell. Although dendritic cells presenting antigen to a T cell can “activate” that T cell, the activated T cell might not be capable of mounting an effective immune response. Effective immune response by the CD8⁺ T cell often requires prior stimulation of the DC by one or more of a number of interactions. These interactions include direct contact of a CD4⁺ T cell to the DC (by way of contact the CD4⁺ T cell's CD40 ligand to the DCs CD40 receptor), or direct contact of a TLR agonist to one of the dendritic cell's toll-like receptors (TLRs).

Humoral immunity refers to B cells and antibodies. B cells become transformed to plasma cells, and the plasma cells express and secrete antibodies. Naïve B cells are distinguished in that they do not express the marker CD27, while antigen-specific B cells do express CD27. The secreted antibodies can subsequently bind to tumor antigens residing on the surface of tumor cells. The result is that the infected cells or tumor cells become tagged with the antibody. With binding of the antibody to the infected cell or tumor cell, the bound antibody mediates killing of the infected cell or tumor cell, where killing is by NK cells. Although NK cells are not configured to recognize specific target antigens, in the way that T cells are configured to recognize target antigens, the ability of NK cells to bind to the constant region of antibodies, enables NK cells to specifically kill the cells that are tagged with antibodies. The NK cell's recognition of the antibodies is mediated by Fc receptor (of the NK cell) binding to the Fc portion of the antibody. This type of killing is called, antibody-dependent cell cytotoxicity (ADCC). NK cells can also kill cells independent of the mechanism of ADCC, where this killing requires expression of MHC class I to be lost or deficient in the target cell.

Without wishing to be bound to any particular mechanism, the disclosure encompasses administration of cancer stem cell antigens, or administering dendritic cells loaded with cancer stem cell antigens, where the antigens stimulate the production of antibodies that specifically recognize one or more of the cancer stem cell antigens, and where the antibodies mediate ADCC. The phrase, loaded with antigens, refers to the ability of the dendritic cell to capture live cells, to capture necrotic cells, to capture dead cells, to capture polypeptides, or to capture peptides, and the like. The tumor antigens comprise cell extracts of the OC-CSC, lysates of the OC-CSC, or intact OC-CSC cells. In another embodiment, the tumor antigens comprise messenger RNA transfected into the dendritic cells ex vivo.

Capture by cross-presentation is encompassed by the present disclosure. Also encompassed is the use of antigen-presenting cells that are not dendritic cells, such as macrophages or B cells.

The technique of “delayed type hypersensitivity response” can be used to distinguish between immune responses that mainly involve cellular immunity or mainly involve humoral immunity. A positive signal from the delayed type hypersensitivity response indicates a cellular response.

The present disclosure provides compositions and methods, where tumor cells are inactivated, e.g., by radiation, nucleic acid cross-linkers, polypeptide linkers, or combinations of these. Cross-linking is the attachment of two chains of polymers molecules by bridges, composed of either an element, a group, or a compound that join certain carbon atoms of the chains by primary chemical bonds. Cross-linking occurs in nature in substances made up of polypeptide chains that are joined by the disulfide bonds involving two cysteine residues, as in keratins or insulin, trivalent pyridinoline and pyrrole cross-links of mature collagen, and cross-links in blood clots which involve covalent epsilon-(gamma-glutamyl)lysine cross-links between the gamma-carboxy-amine group of a glutamine residue and the epsilon-amino group of a lysine residue.

Cross-linking can be artificially effected in proteins, either adding a chemical substance (cross-linking agent), or by subjecting the polymer to high-energy radiation. Cross-linking with fixatives and heat-induced aggregation has been shown to enhance immune responses as well as completely inhibit proliferation. Substances that may be used to cross-link proteins on the surface, and therefore prevent proliferation, of OV-CSC include, but are not limited to, 10% neutral-buffer formalin, 4% paraformaldehyde, 1% glutaraldehyde, 0.25-5 mM dimethyl suberimidate, ice-cold 100% acetone or 100% methanol. Additionally, combinations of 1% glutaraldehyde and 4% paraformaldehyde in 0.1 M phosphate buffer solution may also be used.

Formaldehyde and glutaraldehyde have both been shown to induce the activation of T helper type 1 and type 2 cells. In particular, heat induced aggregation of antigens was also shown to enhance the in vivo priming of cytotoxic T lymphocytes. Cross-linking of antigens by 3,3′-dithiobis(sulfosuccinimidylpropionate) results in increased binding of antigens to dendritic cells and the cross-linked antigens are processed through the proteosomal pathway for antigen presentation. Furthermore, formalin fixed ovarian carcinoma tumor cells have been used in clinical trials with no evidence of proliferation.

In one embodiment, whole OV-CSC are fixed with cross-linking agents, and then used as the antigen source in combination with the dendritic cells.

In another embodiment, the nucleic acids of the cells are cross-linked. An exemplary nucleic acid alkylator is beta-alanine, N-(acridin-9-yl), 2-[bis(2-chloroethyl)amino]ethyl ester. Exemplary cross-linkers, such as psoralens, often in combination with ultraviolet (UVA) irradiation, have the ability to cross-link DNA but to leave proteins unmodified. For instance, the nucleic acid targeting compound can be 4′-(4-amino-2-oxa)butyl-4,5′,8-trimethylpsoralen (S-59). Cells can be inactivated with 150 μM psoralen S-59 and 3 J/cm² UVA light (FX 1019 irradiation device, Baxter Fenwal, Round Lake, Ill.). The inactivation with S-59 with UV light is referred to as photochemical treatment, where treatment conditions can be adjusted or titrated to cross-linked DNA to the extent that cell division is completely prevented, but where damage to polypeptides, including polypeptide antigens, is minimized. Cells can be suspended in 5 mL of saline containing 0, 1, 10, 100, and 1000 nM of psoralen S-59. Samples can be UVA irradiated at a dose of approximately 2 J/cm². Each sample can then transferred to a 15 mL tube, centrifuged, and the supernatant removed, and then washed with 5 mL saline, centrifuged and the supernatant removed and the final pellet suspended in 0.5 mL of saline. See U.S. Pat. Nos. 7,833,775 and 7,691,393, which are incorporated herein by reference for all they disclose regarding inactivation of cells.

For any cell preparation that is treated with a cross-linking agent, the ability to divide can be tested by the skilled artisan by incubating or culturing in a standard medium for at least one week, at least two weeks, at least three weeks, at least four weeks, at least five weeks, at least two months, at least three months, at least four months, and so on. Cell division can be assessed by stains that reveal chromosomes, and that reveal that cell division is, or is not, taking place. Cell division can also be measured by counting cells. Thus, where the number of cells in a culture plate remains stable for a period of two weeks, one month, or two months, and so on, it can reasonably be concluded that the cells cannot divide.

In one embodiment, the dendritic cell immunogenic composition is administered subcutaneously (SC). In further embodiments, each dose ranges from about 5-20 million loaded DCs, repeated in a series of 6-10 doses. In certain embodiments, the doses are administered every five days, every week, every 10 days, every other week, or every third week for two, three, four, five or six doses, followed by administration of doses every two weeks, every three weeks, every four weeks, every month, every five weeks, or every 6 weeks for two, three, four, five or six doses additional doses for a total of 6-10 doses. In one embodiment, the first four injections are given every week for a month, and then once a month for the next 4 injections. In alternative embodiment, administration is once a week for 3 weeks then once a month for 5 months for a total of 8 administrations.

Each dose comprises about 5-20×10⁶ loaded DCs, about 5-17×10⁶ loaded DCs, about 6-16×10⁶ loaded DCs, about 7-15×10⁶ loaded DCs, about 7-14×10⁶ loaded DCs, about 8-13×10⁶ loaded DCs, about 8-12×10⁶ loaded DCs, or about 9-11×10⁶ loaded DCs. In additional embodiment, each dose comprises about 8×10⁶ loaded DCs, about 9×10⁶ loaded DCs, about 10×10⁶ loaded DCs, about 11×10⁶ loaded DCs, or about 12×10⁶ loaded DCs. The loaded DCs comprise a mixture of DCs and residual OV-CSCs which have not been taken up by the DCs. The administered dose comprises a mixture of these cells and the dose reflects this mixture.

In another embodiment, the loaded DCs are administered with a pharmaceutically acceptable carrier or excipients. The pharmaceutically acceptable excipients described herein, for example, vehicles, adjuvants, carriers or diluents, are well-known to those who are skilled in the art and are readily available to the public. It is preferred that the pharmaceutically acceptable carrier or excipient be one which is chemically inert to the loaded DCs and one which has no detrimental side effects or toxicity under the conditions of use.

The choice of excipient or carrier will be determined in part by the particular therapeutic composition, as well as by the particular method used to administer the composition. The formulations described herein are merely exemplary and are in no way limiting.

Often the physiologically acceptable carrier is an aqueous pH buffered solution. Examples of physiologically acceptable carriers include, but are not limited to, saline, solvents, dispersion media, cell culture media, aqueous buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

In some exemplary implementations, an adjuvant is given simultaneously with every dose. In certain embodiments, the cell dose is suspended in a carrier containing an adjuvant. In alternative exemplary implementations, an adjuvant is administered, but not with every single dose. In other exemplary implementations, there is no adjuvant at all. In one embodiment, the adjuvant is GM-CSF.

Without limitation, dendritic cells (e.g., autologous or allogeneic dendritic cells) are contacted with cancer stem cell antigens as a cell lysate, acid elution, cell extract, partially purified antigens, purified antigens, isolated antigens, partially purified peptides, purified peptides, isolated peptides, synthetic peptides, or any combination thereof. The dendritic cells are then administered to a subject, for example, a subject having OV, or a control subject not having OV. In exemplary implementations, dendritic cells are contacted with, injected into, or administered, by one or more of a route that is subcutaneous, intraperitoneal, intranodal, intramuscular, intravenous, intranasal, inhaled, oral, by application to intestinal lumen, and the like. Additionally, the immunogenic compositions can be administered directly to the site of a tumor or metastasis.

### EXAMPLES

### Example 1

### Generation of Cancer Stem Cell Lines from Ovarian Tumor Samples

Samples of ovarian tumors from 3 patients were obtained during surgical procedure after proper consenting. The samples were transported to the tissue processing facility in media containing antibiotics and maintained at a temperature of 4-10° C. during transportation.

The samples were minced with surgical blades in a sterile Petri dish then the fragments exposed to a solution containing collagenase-I, 3000 UI to 6000 UI/gram of tissue, depending on the tissue consistency, under continuous agitation.

The dissociated cells were washed in media, counted and frozen in aliquots for later use in RPMI media containing 15% FBS and 10% DMSO.

The typical aliquots contain 10-30×10⁶ cells and the expected viability at thaw is 40% to 75%. The aliquots can be utilized for later purification and expansion of the cancer stem cells.

### Example 2

### Optimal Media Composition for OV-CSC Isolation and Expansion

Three commercially available ovarian cancer cell lines were compared to cryopreserved tumor cells dissociated from 3 different samples obtained from ovarian cancer patients.

The spherogenic property of the cancer stem cells was used to isolate cancer stem cells. Two different media as shown in Table 5 consisting of a literature described composition (Lit-M) and a proprietary formulation (CSC-M) were used to assess the spherogenic properties of the tumor lines.

OVCAR-3, SKOV-3 and TOV-21G commercial cell lines and the enzyme digested tumors from patients were thawed from master cell bank. After a count and viability evaluation, 50,000 viable cells/cm² from each line were seeded in appropriately labeled ultra-low adherent flasks (Corning).

The cultures were grown for 7 days and media replaced every second day or Monday-Wednesday-Friday schedule. To feed the cultures, the cell suspension was collected in 50 mL tubes, centrifuged at 150 rcf for 3 minutes, and the supernatant replaced with fresh CSC-M or Lit-M media.

After 7 days, the cells were transferred into regular cell culture flasks and media was changed to Lit-E and CSC-E (expansion) formulations (Table 5). CSC-E medium is expansion media and is serum containing; CSC-M is serum-free selection medium. Both CSC-E and CSC-M are based on the basal media of Tables 2-4. The spheroids were dissociated first with collagenase/hyaluronidase mix for 5-10 minutes and gently pipetting, then the enzyme was removed by centrifugation and replaced with media. The cells were expanded for 2 passages following the M-W-F feeding schedule, re-plating each time at a density of 10,000 cells/cm². Finally the cells were transferred into 96 well plates for immunocytochemistry analysis at a density of 5,000-10,000 cells/well. After 2 days the plates were fixed with 4% paraformaldehyde and stained for the following markers: Nanog, Nestin, Sox2, CD133, CD117, NCAM, EpCAM, Slug/Snail, CD24, CA-125, ALDH, CD46, CEA, He-4, Muc-1, CD44, and HER-2.

TABLE 5
Media formulations used in purification and expansion of OV-CSC

| Media | Description | Component | Concentration | |
|---|---|---|---|---|
| Lit-M | Literature-based | RPMI | 500 | mL |
| | Microsphere generation | insulin | 5 | μg/ml |
| | media | EGF | 10 | ng/ml |
| | | bFGF | 10 | ng/ml |
| | | bovine serum albumin | 0.3% | w/v |
| Lit E | Literature-based | RPMI | 85% | v/v |
| | Expansion media | FBS | 15% | v/v |
| CSC-M | CSC Tumor Microsphere | DMEM:F12 (Table 2) | 450 | mL |
| | generation media | Lineage supplement | 50 | mL |
| | (selection media) | (Table 3) | | |
| | | EGF | 10 | ng/ml |
| | | bFGF | 10 | ng/ml |
| CSC-E | CSC Tumor Expansion | CSC-M | 85% | v/v |
| | media | FBS | 15% | v/v |

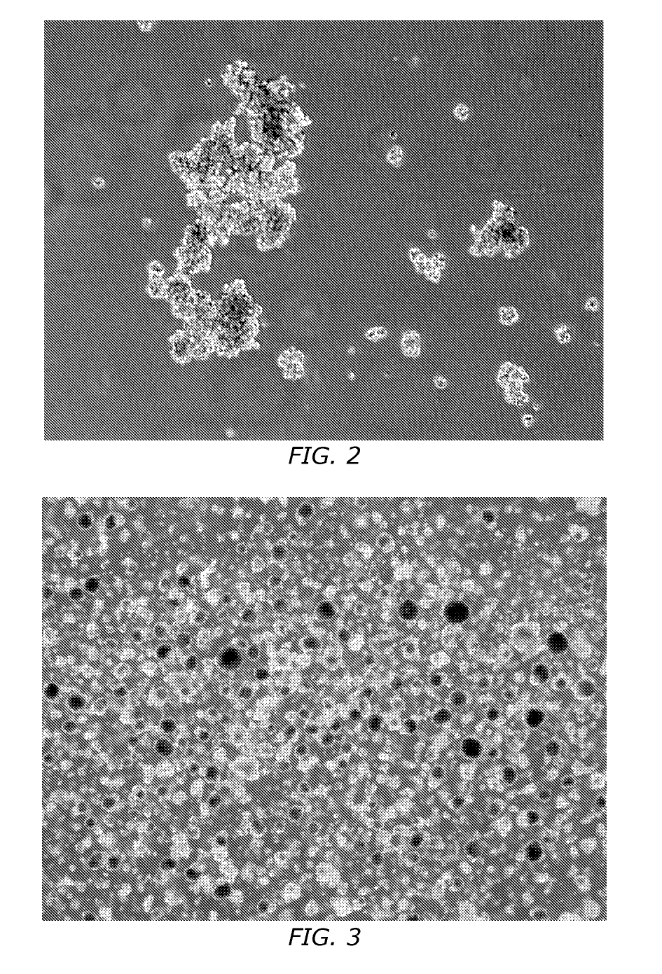

At the end of the 7 days of growth in ultra-low adherent conditions, the commercial cell lines did not form typical cancer stem cell spheroids, in either Lit-M or CSC-M media. Instead they formed loose cell agglomerations with irregular shapes (FIG. 2). The patient tumors formed typical, compact, spherical structures reassembling stem cell spheroids (FIG. 3) that progressively expanded over the 7 days.

After transferring the cells in adherent conditions, cultures expanded slowly reassembling a monolayer of epithelial cells. Some of the cells displayed fast growing phenotypes of small cuboidal cells and some shaped as large epithelial cells with vacuolated cytoplasm or small, spindles reassembling mesenchymal cells. The commercial cell lines expanded faster in the CSC-E media as compared to Lit-E condition. The patient lines expanded slower in the identical conditions, distressed when passaged showing cytoplasm vacuoles, detachment from substrate and extensive cell death.

In this experiment we found that commercial cell lines are of a more differentiated and homogenous phenotype, adapted to a high concentration of FBS (15%), but may not contain cancer stem cells demonstrated by the lack of typical spherogenic property. The patient samples instead contained sufficient cancer stem cells to produce the expected compact spheres. Transferred in the 15% serum containing media, the patient lines were distressed compared to the commercial lines and proper immunocytochemical characterization was not possible immediately after sphere plating.

TABLE 6
Summary of ICC scoring for patient
derived ovarian cancer cell lines

| | Patient 411 | | Patient 561 | | Patient 394 | |
|---|---|---|---|---|---|---|
| Marker | % | Intensity | % | Intensity | % | Intensity |
| Muc-1 | + | 1 | ++ | 2 | + | 2 |
| CA125 | ++ | 2 | + | 2 | ++ | 1 |
| EpCAM | + | 2 | ++ | 3 | + | 3 |
| NCAM | +++ | 3 | +++ | 2 | +++ | 3 |
| CD133 | | 0 | + | 1 | | 0 |
| Nestin | +++ | 3 | + | 3 | ++++ | 3 |
| CD117 | +++ | 3 | ++ | 3 | +++ | 2 |
| Slg/Snl | ++++ | 3 | ++++ | 3 | ++++ | 3 |

Reactivity % positive scale: + = 1-25%, ++ = 26-50%, +++ = 51-75%, ++++ = 76-100%.
Intensity scale: 1 = light, 2 = medium, 3 = high

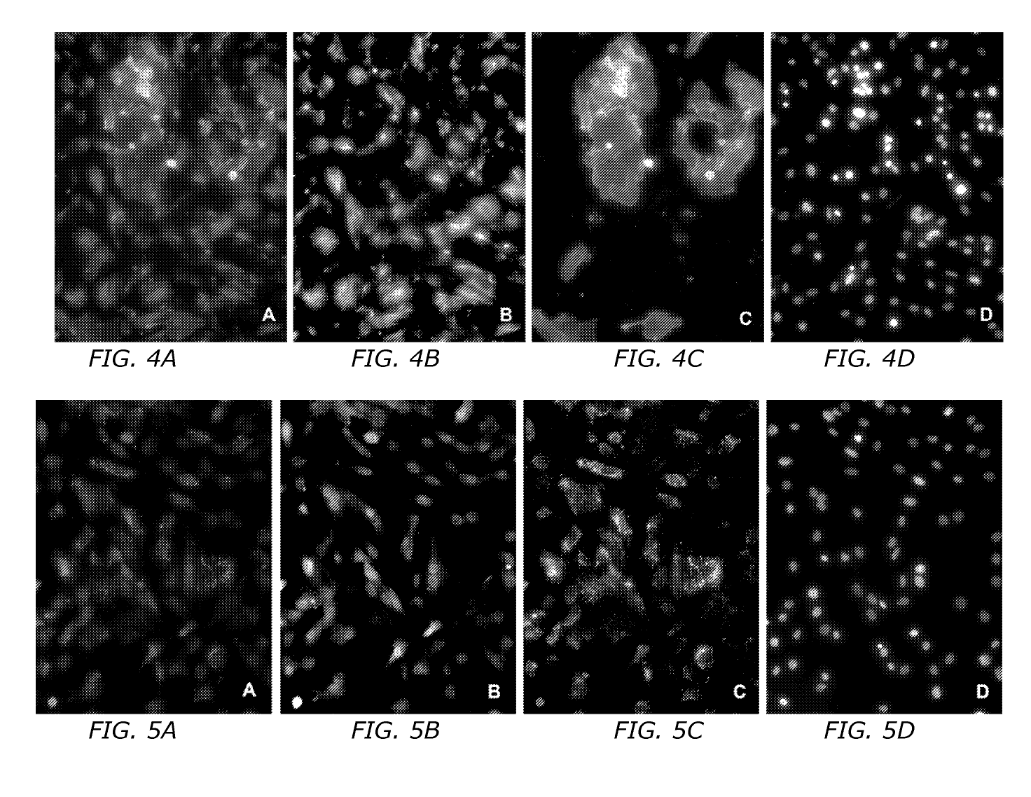

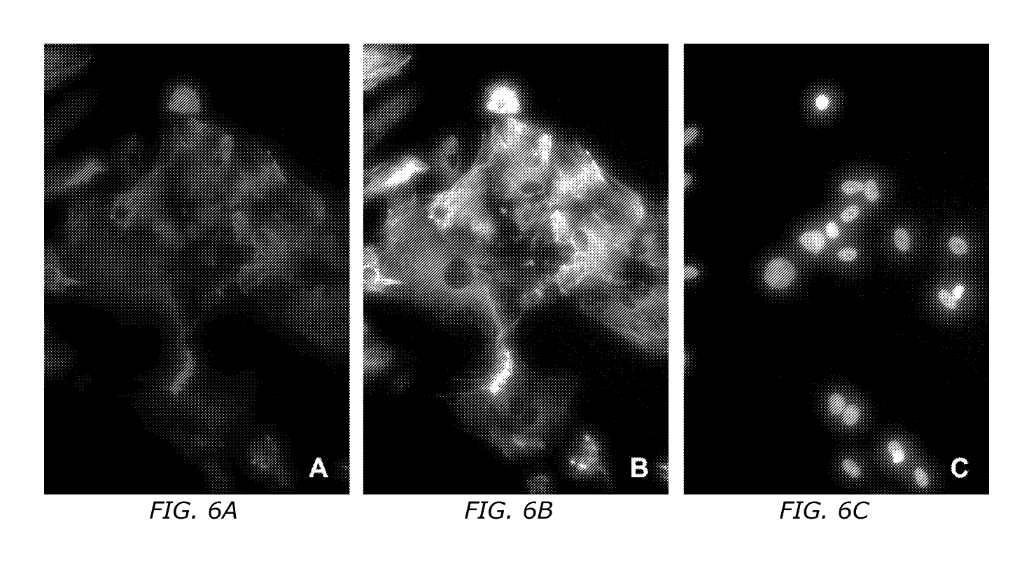

As demonstrated by the ICC results (Table 6), the cultures show EMT phenotype (epithelial to mesenchymal transition): low EpCAM, high NCAM (FIG. 4A-D), Slug/Snail (Slg/Snl), CD117 (FIG. 5A-D) and nestin (FIG. 6A-C)

In conclusion, 1) commercial cell lines are not equivalent to the fresh patient samples regarding the CSC content; 2) the OV-CSC are susceptible to media composition, and physical manipulations and do not tolerate passaging in the tested conditions; 3) the proprietary media formulation (CSC-M and CSC-E) advantaged the growth of the OV-CSC; and 4) the tested culture conditions are favorable for EMT.

### Example 3

### Single Plating Expansion of Tumor Cells

The typical in vitro cell culture is a cyclic process in which cells are plated to a lower density, allowed to grow until reaching confluence, then dissociating and seeding again at low density on larger surface (passaging). Our previous observations concluded that OV-CSCs are sensitive to physical and enzymatic manipulation during passaging manifested by differentiation and extensive cell death and epithelial to mesenchymal transition (EMT). To overcome this phenomenon, in the next experiment we tested the possibility of a single passage expansion of the OV-CSC, to seed the cells at a low density on the final surface that can provide the total cell number required for active specific immunotherapy (ASI). In common cell culture conditions, the single expansion is avoided to save the culture space in the incubators and stimulate the cell proliferation by periodic enzymatic dissociation and avoidance of contact inhibition.

In this approach, the cells were plated at various low densities of 5000, 10,000, and 15,000 cells/cm² and allowed to expand for 4 days. Cultures were fed on a Monday-Wednesday-Friday feeding schedule with CSC-E media (Table 5) that contained only 5% FBS instead of 15% in the initial formulation. At the end, the cells were counted and analyzed for phenotype.

The cell counts in the various conditions did not show significant difference regarding the doubling time. The immuno-cytochemistry (ICC) revealed identical profiles, regardless of initial seeding density (Table 7).

TABLE 7
Summary of ICC scoring

| | Density | | | | | | | |
|---|---|---|---|---|---|---|---|---|
| | 5,000/cm² | | 10,000/cm² | | 15,000/cm² | | 15K | |
| | | Inten- | | Inten- | | Inten- | | Inten- |
| Marker | % | sity | % | sity | % | sity | % | sity |
| Muc-1 | + | 2 | ++ | 2 | ++ | 2 | ++ | 2 |
| CA125 | + | 1 | + | 2 | + | 2 | + | 2 |
| EpCAM | ++ | 3 | ++ | 3 | ++ | 3 | ++ | 3 |
| NCAM | +++ | 2 | +++ | 2 | +++ | 3 | +++ | 2 |
| CD133 | + | 1 | + | 1 | + | 1 | + | 1 |
| Nestin | + | 3 | + | 3 | ++ | 2 | +++ | 3 |
| CD117 | +++ | 3 | ++ | 3 | +++ | 3 | ++ | 3 |
| Slg/Snl | ++++ | 3 | ++++ | 3 | ++++ | 2 | ++++ | 3 |

Reactivity % positive scale: + = 1-25%, ++ = 26-50%, +++ = 51-75%, ++++ = 76-100%
Intensity scale: 1 = light, 2 = medium, 3 = high

In order to avoid the loss of cells or causing differentiation by mechanical or enzymatic dissociation, a method can be used to expand the cells to the desired number by initially seeding at low density and allowing the cultures to growth until confluence is reached without repeated passaging before the final harvesting. Additionally, the media containing 5% FBS caused EMT, according to the ICC profile (Table 7) just like the higher concentration (15%) of serum in the previous experiments (Table 6).

### Example 4

### Determination of Cell Culture Conditions for Selection of Early Ovarian Cancer Stem Cells

As shown in the previous experiments, FBS-containing media causes differentiation of the cancer stem cells and EMT. This phenomenon may be caused by factors contained in the serum such as bone morphogenic protein (BMP4). In cultures containing FBS, or by addition of BMP4, the cells appear larger, with an epithelial look and slower doubling rate that is typical for the well-differentiated carcinoma. If growth factors are added to this media, EMT is observed. In vivo, the ovarian cancer expands almost exclusively in the abdominal cavity where high quantity of growth factors secreted by peritoneal cells could fuel the tumor expansion. Such growth factors include FGF, EGF, VEGF, HGF, PDGF, activin A, and TGFbeta.

The next experiments investigated the media and substrate composition that is permissive for rapid expansion of ovarian cancer stem cells.

Patient-derived ovarian tumor cells were plated on plain tissue culture plastic, 1% gelatin, or MATRIGEL® (1:60). For each substrate condition, various media formulations were used: CSC-E modified with 5% FBS; CSC-M (Table 5); a commercial media formulated for epithelial cells (keratocytes) KGM-GOLD™ (Lonza); and serum-free media formulation that is used for human embryonic stem cell culture (STEMBLAST®). Each condition was tested with or without the addition of growth factors bFGF (10 ng/mL) and EGF (10 ng/mL).

The KGM-GOLD™ basal media is formulated with low calcium for epithelial cell expansion. STEMBLAST® is a serum free formulation used for the cultivation of embryonic stem cells that contains 5 ng/mL activin A. MATRIGEL® is a complex extracellular matrix containing laminin, collagen and proteoglycans.

Morphologic analysis and growth performance was performed after 1 week of cultivation in these conditions and summarized in Table 8.

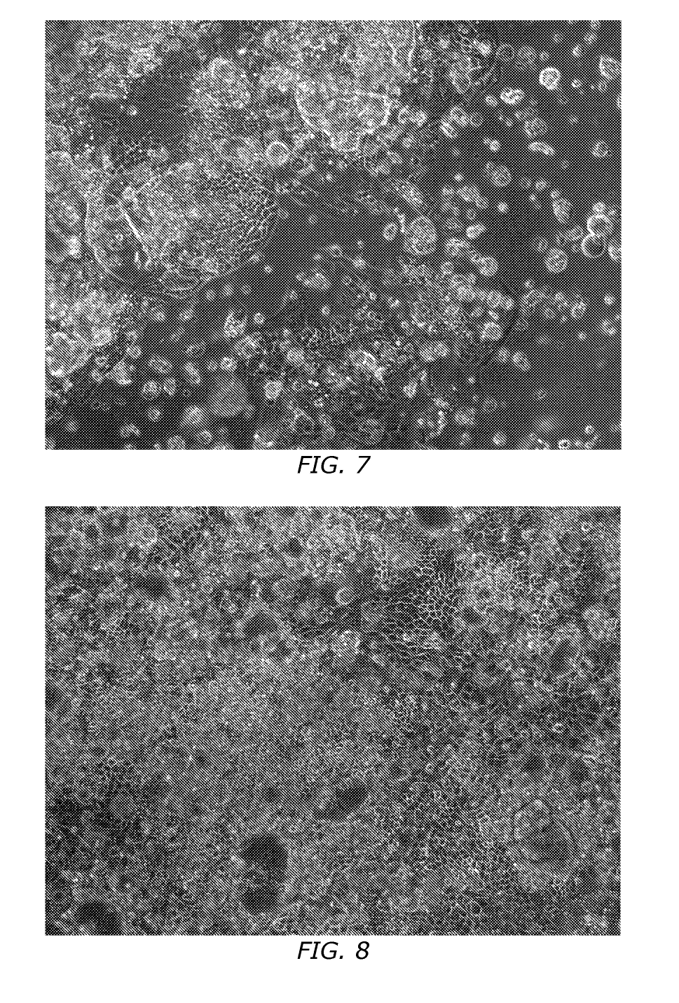

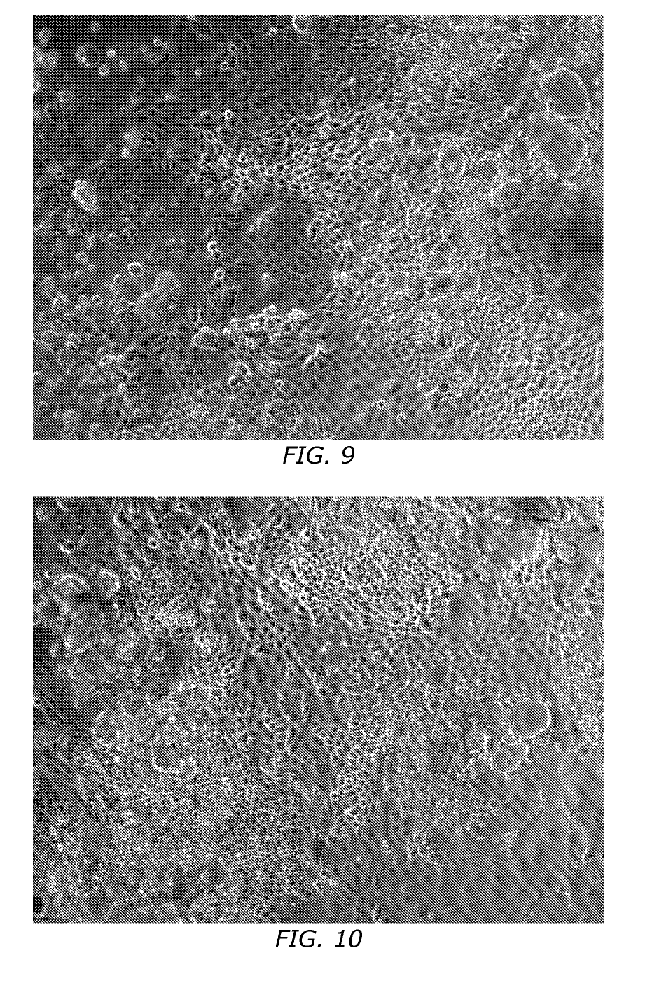

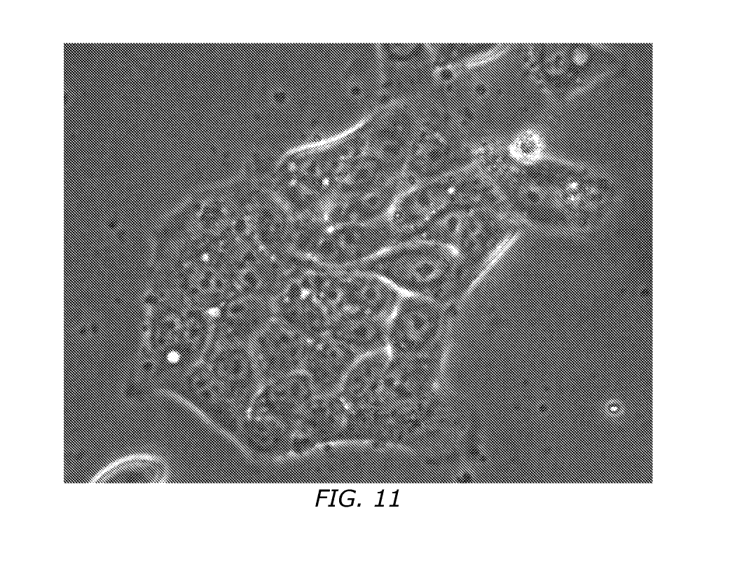

TABLE 8
Patient derived ovarian cancer culture performance in various media, substrate and
growth factors conditions

| Media/Substrate | Tissue culture plastic | Gelatin 0.1% | MATRIGEL® |
|---|---|---|---|
| CSC-E (5% FBS) | Expanding slow, in | Better initial attachment. | Not expanding, or |
| | colonies, many loose | Expanding slow, in | very slow, small |
| | cells and debris, cells | colonies, many loose cells | colonies, many loose |
| | with vacuolated | and debris, cells with | cells and debris, cells |
| | cytoplasm | vacuolated cytoplasm | with vacuolated |
| | | (FIG. 7) | cytoplasm |
| CSC-E (5% FBS) + | Rapid expansion, large | Better initial attachment. | Slow expansion, |
| bFGF + EGF | colonies with | Rapid expansion, large | small colonies, many |
| | heterogenous | colonies with | loose cells and |
| | morphologies. Areas of | heterogenous | debris, cells with |
| | small cuboid monolayers | morphologies. Areas of | vacuolated cytoplasm |
| | mixed with large | small cuboid monolayers | |
| | epithelial cells with | mixed with large epithelial | |
| | vacuolated cytoplasm. | cells with vacuolated | |
| | | cytoplasm. (FIG. 8) | |
| CSC-M | Good expansion, | Better initial attachment. | Slow expansion, |
| | homogenous colonies | Good expansion, | colonies with larger, |
| | with cuboid cells, few | homogenous colonies with | vacuolated cells. |
| | vacuolated large | cuboid cells, Some | |
| | epithelial cells, less | colonies with larger | |
| | debris. | epithelial cells. Few | |
| | | vacuolated large epithelial | |
| | | cells, less debris. (FIG. 9) | |
| CSC-M + FGF + | Good expansion, | Better initial attachment. | Slow expansion, |
| EGF | comparable with the | Faster expansion than the | colonies with larger, |
| | gelatin substrate | growth factor free | vacuolated cells. |
| | condition. | condition. Homogenous | |
| | | colonies with cuboid cells, | |
| | | Some colonies with larger | |
| | | epithelial cells few | |
| | | vacuolated large epithelial | |
| | | cells, less debris. (FIG. 10) | |
| KGM-GOLD™ | Very slow or not growing, | Very slow or not growing, | Very slow or not |
| | many debris | many debris | growing, many debris |
| KGM-GOLD™ + | Slow growing | Better initial attachment, | Slow growing |
| FGF + EGF | differentiated colonies | slow growing differentiated | differentiated colonies |
| | | colonies | |
| STEMBLAST® | Slow growing compact | Slow growing compact | Not growing |
| | colonies with small cells | colonies with small cells | |
| STEMBLAST® + | Compact colonies. Few | Good expansion of | Slow growing |
| FGF + EGF | differentiated, large | compact colonies with | compact colonies. |
| | epithelial cells. Minimal | small cuboidal cells and | Few differentiated, |
| | debris | large nucleus. Few | large epithelial cells. |
| | | differentiated large or | Minimal debris |
| | | vacuolated cells, minimal | |
| | | debris (FIG. 11) | |

MATRIGEL® inhibited the growth of tumor cells in all media or growth factor conditions. The tissue culture plastic (plasma treated) displayed less adherence of the cells at initial plating, however did support adequate expansion of the tumor cells in all conditions. Gelatin (0.1%) coating allowed very good initial adherence and supported fast expansion of the tumor cells.

KGM-GOLD™ did not support a fast expansion of the tumor cells, regardless of the substrate or presence of growth factors, caused senescence of the ovarian cancer cells that appeared with large cell bodies and vacuolated cytoplasm.

The CSC-E media containing 5% FBS supported fast expansion of the cells only in the presence of bFGF and EGF (FIGS. 7 and 8), while the serum free version supported the fast expansion regardless of the presence of the growth factors (FIGS. 9 and 10). It appears the animal serum contain factors that are inhibiting the fast expansion of the ovarian tumor cells, effect that is compensated by the addition of growth factors that are ligands to receptor tyrosine kinases (FGF, EGF). Other ligands to receptor tyrosine kinases (RTK) such as VEGF, HGF, PDGF may have the same effect on the tumor cell expansion. The morphology of the cells grown in serum free media that is supplemented with FGF and EGF is of a small, cuboid type, packed in dense and homogenous colonies resembling embryonic stem cell cultures.

STEMBLAST® supported good expansion only in the presence of the growth factors. The association of EGF, FGF and activin A in STEMBLAST® caused a very similar morphology to embryonic stem cell cultures with small cell bodies and large nucleus that occupies the majority of the cell and minimal cytoplasm (FIG. 11).

Immunocytological analysis for the common ovarian and cancer stem cell markers in the serum-free media revealed a cancer stem cell phenotype and negligible EMT.

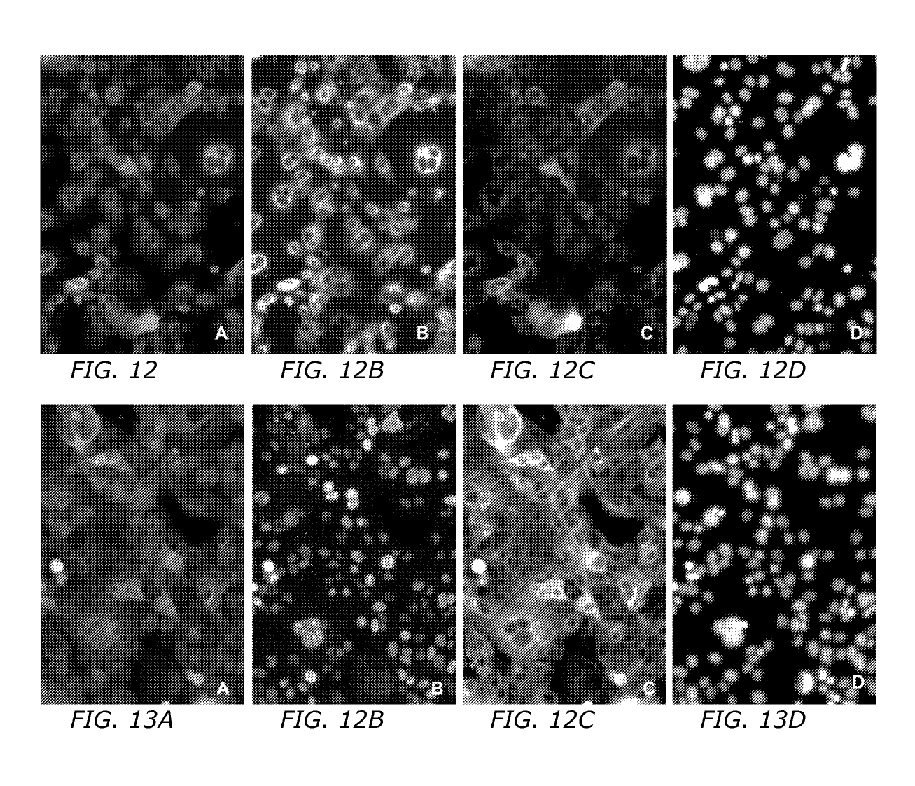

The ovarian cancer epithelial markers marker CA125 was found on more than 90% of the cells (FIG. 12 B). MUC-1, another ovarian cancer epithelial marker was found in about 60% of cells with various intensity of expression (FIG. 12C). Ovarian cancer cytokeratin marker CK8 was present in more than 80% of the cells (FIG. 13B).

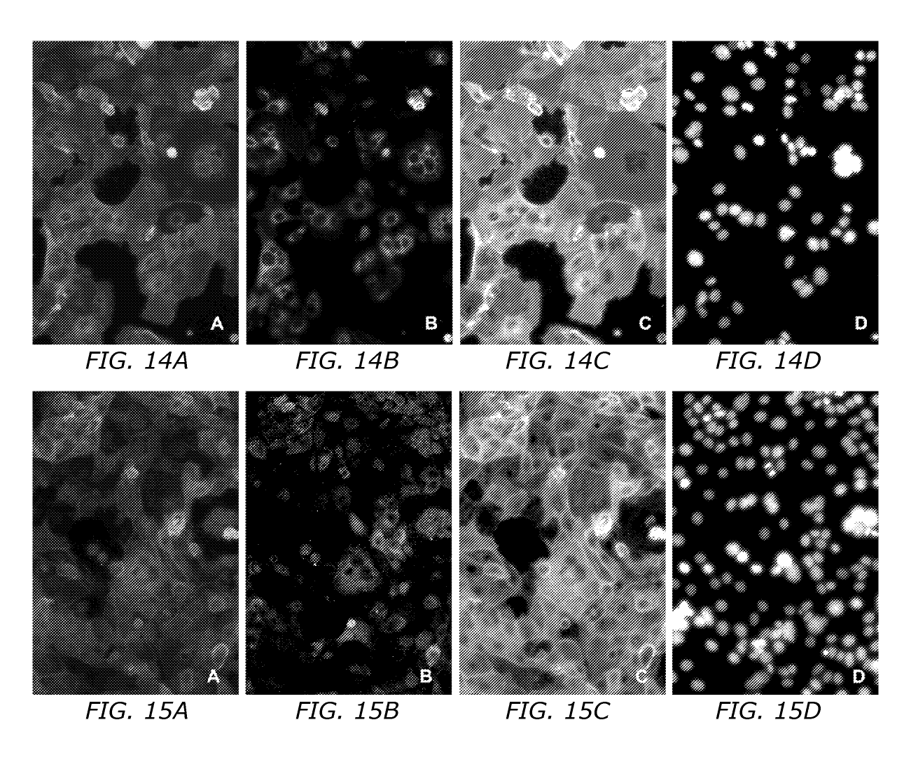

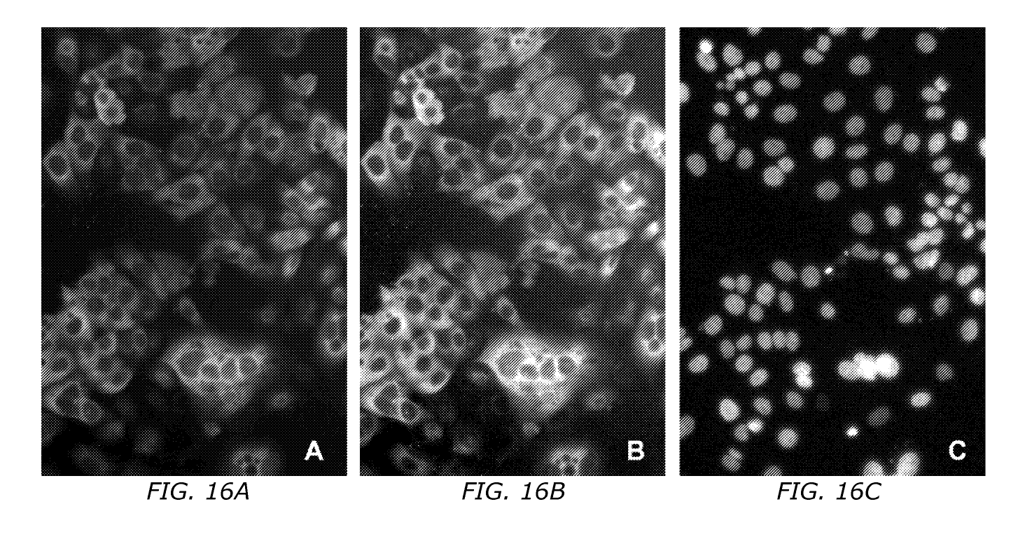

The culture conditions promoted the expansion of cancer stem cells, demonstrated by the presence in high percentage (better than 75%) of cells expressing EpCAM (FIG. 14C), CD44 (FIG. 15C), and CD133 (FIG. 16). The cultures show a rapid expansion by the high level expression of the Ki67 proliferative marker (FIG. 13B) on the majority of the cells.

We concluded that a serum free media with minimal amount of or ligands for RTK or low concentration animal serum in media supplemented with ligands for RTK (such as FGF, EGF) can maintain and expand optimally a population of ovarian cancer stem cells. Increasing the amount of serum and decreasing the RTK ligands will cause differentiation of the CSC. Increasing the serum concentration and increasing the RTK ligand concentration will cause EMT. A serum free media containing RTK ligand and activin A can sustain a very early stem cell status, embryonic stem cell-like.

Although the presence of a gelatin (collagen) coating increased the initial cell adherence, it did not significantly change the expansion rate or the phenotype of the cells.

These experiments demonstrate our ability to isolate, purify and expand CSC from ovarian tumors and manipulate for differentiation or EMT. The cell populations described here can be used as high quality antigenic sources for a whole cell active autologous immune therapy, compared to the diluted antigenicity of the bulk tumors.

### Example 5

### Production of Loaded Dendritic Cell Compositions

The antigen source is autologous tumor cells from continuously proliferating, self-renewing cells derived from the patient's fresh tumor tissue. These cells have the characteristics of tumor stem cells. At all times in the surgical and pathology setting, biopsies are handled with strict adherence to sterility protocols to ensure that samples are sterile.

The pathologist obtains fresh tissue from biopsy of the patient's tumor. Using sterile scalpels and forceps, the specimen is cut into 10 mm slices and transferred to the transport tubes containing transport media, working quickly to avoid specimen drying. Specimens are shipped by overnight courier to the manufacturing facility within 48 hours of surgical resection.

At the manufacturing facility, samples are dissociated into single cell suspensions in a clean room and placed in cell culture conditions designed to enrich for and proliferate the OV-CSC. During the processing of the tumor specimen, normal cells such as lymphocytes, stromal cells and connective tissue are eliminated. Upon completion of the expansion and purification steps, the enriched proliferating OV-CSC (tumor cells, TC) are inactivated by irradiation and placed in vapor phase liquid nitrogen storage. This process can take up to eight weeks, depending on the quantity and quality of the tumor specimen.

Once the tumor cell product has cleared quality assurance, the patient is notified to undergo a procedure called leukapheresis (usually a six liter procedure). This process entails the filtering of blood to collect peripheral blood mononuclear cells (PBMCs). The collected PBMC are shipped to the manufacturing facility by overnight courier for further purification by counter flow density centrifugation called elutriation. Elutriation is a process by which monocytes are purified from other lymphocytes in order to enrich for cells that can be turned into antigen presenting cells or dendritic cells. To generate the dendritic cells, the elutriated monocytes are incubated with the cytokines GM-CSF and interleukin-4 (IL-4) for six days.

On Day 6, the purified tumor cell product is removed from cryostorage, thawed and combined with the dendritic cells for 18-24 hours. This process results in “antigen loading” of the DC. The final product is either entirely DC or may contain some residual irradiated TC (which is considered permissible), and is referred to as DC-TC. The combined dendritic cell/tumor cell mixture is collected, cryopreserved to retain viability of the dendritic cells and stored in vapor phase liquid nitrogen.

Upon completion of the quality controls assays and release of the autologous cell therapy product, the batch is shipped to the treatment facility under vapor phase liquid nitrogen conditions. After arrival, the cell therapy product is stored under vapor phase liquid nitrogen conditions until prepared for administration.

### Example 6

### Phase II, Double-Blind, Randomized, Clinical Trial of Ovapuldencel-T vs. Autologous Peripheral Blood Mononuclear Cells in GM-CSF in Patients with Ovarian Carcinoma

This study is a Phase II, double-blind, randomized, single center trial of ovapuldencel-T (autologous dendritic cells loaded with irradiated autologous OV-CSC in GM-CSF) vs. autologous peripheral blood mononuclear cells In GM-CSF (MC) as a component of maintenance or secondary therapy in patients with stage III or IV epithelial ovarian, fallopian tube or primary peritoneal carcinoma after debulking surgery and adjuvant chemotherapy. The purpose of the study is to compare overall survival (OS) from the date of randomization for patients treated with ovapuldencel-T to patients treated with autologous blood mononuclear cells in GM-CSF control. Ovapuldencel-T is prepared according to the methods of Example 5.

Female subjects 18 years or older with newly diagnosed stage III or IV ovarian cancer, who are candidates for surgical debulking and receipt of chemotherapy are candidates for the study. Performance status must be an ECOG score of 0 or 1 at the time of enrollment for ASI treatment.

Inclusion criteria include histologic diagnosis of epithelial ovarian, fallopian tube or primary peritoneal carcinoma; advanced (metastatic, stage III or IV) epithelial ovarian, fallopian tube or primary peritoneal carcinoma and a candidate for surgical debulking to obtain fresh viable tumor tissue for efforts to establish a short-term tumor cell line; age >18 years; each patient must be aware of the neoplastic nature of her disease process and must willingly consent to the manipulation of tumor tissue for efforts to establish a tumor cell line; and patients must have the ability and willingness to travel to the treatment center for administration of ASI treatment.

Exclusion criteria includes ECOG performance status greater than 2; known positive for hepatitis B or C or HIV; pregnant or lactating women; underlying cardiac disease associated with myocardial dysfunction that requires active medical treatment, or unstable angina related to atherosclerotic cardiovascular disease, or under treatment for arterial or venous peripheral vascular disease; diagnosis of any other invasive cancer which is considered to be life-threatening within the next five years, and/or taking anti-cancer therapy for cancer other than ovarian (such as continuation of hormonal therapy for prostate or breast cancer diagnosed more than five years earlier); active infection or other active medical condition that could be eminently life-threatening, including active blood clotting or bleeding diathesis; active central nervous system metastases at the time of treatment; known autoimmune disease, immunodeficiency, or disease process that involves the use of immunosuppressive therapy; a low malignancy potential tumor; or received another investigational drug within 28 days of the first dose.

Approximately 99 patients with stage III or IV epithelial ovarian, fallopian tube or intraperitoneal cancer for whom a cell line has been successfully established and who are eligible for ASI treatment at the time of randomization and who have granted consent for participation in the trial and treatment with ASI will be enrolled.

After patients have recovered from debulking surgery and prior to initiating chemotherapy, patients will undergo leukapheresis to obtain PBMC from which autologous dendritic or mononuclear cells are derived. Following leukapheresis, patients will undergo a planned 6 cycles of primary adjuvant chemotherapy that includes a taxane and a platinum chemotherapy agent, per standard of care for such patients.

Patients will be stratified into: (1) platinum-resistant, based on progression during adjuvant chemotherapy or detectable disease at the conclusion of adjuvant therapy; or (2) platinum-sensitive, with no evidence of disease (NED) at the conclusion of adjuvant therapy per elevated blood CA-125 and/or other tumor markers and/or detection of disease by physical examination or imaging.

Following stratification, patients will be randomized into ovapuldencel-T or MC treatment arms at a 2:1 ratio. This approach creates two clinically similar patient populations, standardizes the timing of therapy, maximizes the use of cell lines, and incorporates a standard of practice with regard to maintenance or secondary adjuvant therapy:

Patients with platinum-resistant disease will be treated with secondary therapy per their managing physician and concurrently will receive ovapuldencel-T or MC treatment.

Patients who are platinum-sensitive with NED will receive maintenance therapy per their managing physician (typically paclitaxel for up to a year) and concurrently will receive ovapuldencel-T or MC treatment.

Patients randomized into the ovapuldencel-T arm will receive autologous dendritic cells pulsed with autologous irradiated OV-CSC cells to create a patient-specific therapy. Ovapuldencel-T will be administered in 500 micrograms of GM-CSF once a week for three weeks and then every three to four weeks at the time subsequent maintenance or secondary therapy is given, for up to a total of 8 doses of vaccine therapy given over four to six months, depending on the timing of the maintenance or secondary therapy.

Patients randomized into the MC arm will receive autologous mononuclear cells administered in 500 micrograms of GM-CSF once a week for three weeks and then every three to four weeks at the time subsequent maintenance or secondary therapy is given, for up to a total of 8 doses of ASI therapy given over four to six months, depending on the timing of the maintenance or secondary therapy.

Patients who experience progressive disease while on study may continue the ASI in conjunction with any other standard systemic therapy as prescribed by their managing oncologist, until all 8 treatment doses have been administered.

The primary study objective is to compare overall survival (OS) from the date of randomization for patients treated with ovapuldencel-T to patients treated with autologous blood mononuclear cells in GM-CSF control.

The primary endpoint of this trial is death from any cause with the metric of overall survival (OS) from the date of randomization. Progression-free survival (PFS) is a secondary endpoint and is calculated as the time from the date of randomization for treatment until subjective tumor progression or death. Progression is subjectively defined by the treating physician, and is based on tumor marker levels (e.g. CA-125) and/or imaging. Secondarily, PFS and OS are defined from the date of debulking surgery.

All patients who qualify for participation in the study will be followed for up to 5 years from date of randomization or until death, whichever occurs first.

Kaplan-Meier curves will display survival times for each treatment group. The log rank test will be used to analyze OS to test the null hypothesis of no treatment difference. The Cox regression model and the Wald test will be used to estimate the hazard ratio associated with treatment and to identify the significance of potential prognostic factors and their impact, if any on the treatment differences. Analyses based on the intention to treat population will be considered as the primary analysis, analyses based on the Per-Protocol population will be considered as a sensitivity analysis. Subgroup analyses of the OS endpoint will mimic the plan laid out for the primary endpoint for platinum-resistant patients and platinum-sensitive patients separately.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Thus, while there have shown and described and pointed out fundamental novel features of the disclosure as applied to an exemplary implementation and/or aspects thereof, it will be understood that various omissions, reconfigurations and substitutions and changes in the form and details of the exemplary implementations, disclosure and aspects thereof may be made by those skilled in the art without departing from the spirit of the disclosure and/or claims. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or implementation may be incorporated in any other disclosed or described or suggested form or implementation as a general matter of design choice. It is the intention, therefore, to not limit the scope of the disclosure. All such modifications are intended to be within the scope of the claims appended hereto.

All publications, patents, patent applications, references, and sequence listings, cited in this specification are herein incorporated by this reference as if fully set forth herein.

The Abstract is provided to comply with 37 CFR §1.72(b) to allow the reader to quickly ascertain the nature and gist of the technical disclosure. The Abstract is submitted with the understanding that it will not be used to interpret or limit the scope or meaning of the claims.

1. An immunogenic composition comprising dendritic cells activated ex vivo by tumor antigens derived from a population of purified ovarian carcinoma cancer stem cells (OV-CSCs).

2. The immunogenic composition of claim 1, wherein the tumor antigens comprise cell extracts of the OV-CSCs.

3. The immunogenic composition of claim 1, wherein the tumor antigens comprise lysates of the OV-CSCs.

4. The immunogenic composition of claim 1, wherein the tumor antigens comprise intact OV-CSCs.

5. The immunogenic composition of claim 4, wherein the intact OV-CSCs are rendered non-proliferative.

6. The immunogenic composition of claim 5 wherein the intact OV-CSCs are rendered non-proliferative by irradiation.

7. The immunogenic composition of claim 5, wherein the intact OV-CSCs are rendered non-proliferative by exposure of the cells to a nuclear cross-linking agent.

8. The immunogenic composition of claim 1, further comprising a pharmaceutically acceptable carrier or excipient.

9. The immunogenic composition of claim 1, further comprising an adjuvant.

10. The immunogenic composition of claim 9, wherein the adjuvant is granulocyte macrophage colony stimulating factor.

11. The immunogenic composition of claim 1, wherein the composition comprises activated dendritic cells and OV-CSCs.

12. The immunogenic composition of claim 1, wherein the OV-CSCs are in form of OV-CSC spheroids.

13. The immunogenic composition of claim 1, wherein the OV-CSCs are early OV-CSCs.

14. The immunogenic composition of claim 1, wherein the OV-CSCs are mixed OV-CSCs.

15. The immunogenic composition of claim 1, wherein the OV-CSCs are EMT-OV-CSCs.

16. A method of treating ovarian carcinoma in a subject in need thereof, comprising administering an immunogenic dose of an immunogenic composition comprising dendritic cells activated ex vivo by tumor antigens derived from a population of purified ovarian carcinoma (OV) cancer stem cells (OV-CSCs) to the subject.

17. The method of claim 16, wherein the immunogenic composition is administered in a plurality of doses, each dose comprising about 5-20×10⁶ cells.

18. The method of claim 17, wherein the dose comprises about 10×10⁶ cells.

19. The method of claim 16, wherein the dose is administered weekly for 2-5 doses, followed by monthly for 3-6 doses.

20. The method of claim 16, wherein the subject receives from 6-10 doses of immunogenic composition.

22. (canceled)

23. (canceled)

24. A method for preparing a population of ovarian carcinoma cancer stem cells (OV-CSCs), the method comprising:
acquiring a sample of an ovarian carcinoma tumor comprising ovarian carcinoma tumor cells;
dissociating the cells of the sample, and
in vitro culturing the dissociated cells in a defined medium on a non-adherent substrate, wherein the defined medium is serum free and is supplemented with at least one growth factor that acts through the mitogen activated protein kinase (MAPK) pathway, thereby forming a population of OV-CSC spheroids;
the OV-CSC spheroid population being characterized by at least 80% of the cells in the OV-CSC spheroid population expressing two or more of the biomarkers EpCAM, CA-125, MUC-1, CD117, He-4, ALDH, CD133, CD24, and Ki-67.

25. The method of claim 24, the OV-CSC spheroid population being characterized by at least 80% of the cells in the OV-CSC spheroid population further expressing one or more of the biomarkers CA19-9, HER2/neu, NCAM, ganglioside CD2, estrogen receptor alpha, vimentin, CK8, CK18, AFP, testosterone, TGFβR, EGFR, TAG-72, CD46, CD44, ABCG2, Slug/Snail, nestin, and TP53.

26. The method of claim 24, the OV-CSC spheroid population being characterized by at least 90% of the cells in the OV-CSC spheroid population expressing two or more of the biomarkers EpCAM, CA-125, MUC-1, CD117, He-4, ALDH, CD133, CD24, and Ki-67.

27. The method of claim 24, further comprising:
culturing the OV-CSC spheroids in a defined medium on an adherent substrate, wherein the defined medium is serum free and is supplemented with at least one growth factor that acts through the MAPK pathway, thereby forming a population of early OV-CSCs, the population of early OV-CSCs being characterized by at least 80% of the cells in the early OV-CSC population expressing two or more of the biomarkers EpCAM, CD133, CD44, Nanog, Sox2, Oct3/4, CD17, and Ki-67.

28. The method of claim 27, the population of early OV-CSCs being characterized by at least 80% of the cells in the early OV-CSC population further expressing one or more of the biomarkers CA-125, MUC-1, TGFβR, and CD24.

29. The method of claim 27, the population of early OV-CSCs being characterized by at least 90% of the cells in the early OV-CSC population expressing two or more of the biomarkers EpCAM, CD133, CD44, Nanog, Sox2, Oct3/4, CD17, and Ki-67.

30. The method of claim 24, further comprising:
culturing the OV-CSC spheroids in a defined medium on an adherent substrate, wherein the defined medium contains serum, thereby forming a population of mixed OV-CSCs, the population of mixed OV-CSCs being characterized by at least 80% of the cells in the mixed OV-CSC population expressing two or more of the biomarkers EpCAM, CA-125, MUC-1, CD117, CK8, CK18, and Ki-67.

31. The method of claim 30, wherein the defined medium further comprises at least one growth factor that acts through the MAPK pathway.

32. The method of claim 30, the population of mixed OV-CSCs being characterized by at least 80% of the cells in the mixed OV-CSC population further expressing one or more of the biomarkers CA19-9, HER2/neu, NCAM, ganglioside CD2, estrogen receptor alpha, testosterone, TGFβR, EGFR, TAG-72, CD46, He-4, ALDH, CD133, CD44, ABCG2, nestin, and TP53.

33. The method of claim 30, the population of mixed OV-CSCs being characterized by at least 90% of the cells in the mixed OV-CSC population expressing two or more of the biomarkers EpCAM, CA-125, MUC-1, CD117, CK8, CK18, and Ki-67.

34. The method of claim 24, further comprising:
culturing the OV-CSC spheroids in a defined medium on an adherent substrate, wherein the defined medium contains serum and is supplemented with at least one growth factor that acts through the MAPK pathway, thereby forming a population of epithelial to mesenchymal transitioned (EMT)-OV-CSCs, the population of EMT-OV-CSCs being characterized by at least 80% of the cells in the EMT-OV-CSC population expressing two or more of the biomarkers NCAM, Slug/Snail, CD24, and Twist.

35. The method of claim 34, the population of EMT-OV-CSCs being characterized by at least 80% of the cells in the EMT-OV-CSC population further expressing one or more of the biomarkers CA-125, MUC-1, CD133, Nanog, CD117, N-cadherin, CD44, and vimentin.

36. The method of claim 34, the population of EMT-OV-CSCs being characterized by at least 90% of the cells in the EMT-OV-CSC population expressing two or more of the biomarkers NCAM, Slug/Snail, CD24, and Twist.

37. The method of claim 24, further comprising:
culturing the OV-CSC spheroids in a defined medium on an adherent substrate, wherein the defined medium is serum free and is supplemented with at least one growth factor that acts through the MAPK pathway, thereby forming a population of early OV-CSCs, the population of early OV-CSCs being characterized by at least 80% of the cells in the early OV-CSC population expressing two or more of the biomarkers EpCAM, CD133, CD44, Nanog, Sox2, Oct3/4, CD17, and Ki-67.

38. The method of claim 37, the population of early OV-CSCs being characterized by at least 80% of the cells in the early OV-CSC population further expressing one or more of the biomarkers EpCAM, CD133, CD44, Nanog, Sox2, Oct3/4, CD17, and Ki-67.

39. The method of claim 37, the population of early OV-CSCs being characterized by at least 90% of the cells in the early OV-CSC population expressing one or more of the biomarkers EpCAM, CD133, CD44, Nanog, Sox2, Oct3/4, CD17, and Ki-67.

40. The method of claim 24, further comprising:
culturing the OV-CSC spheroids in a defined medium on an adherent substrate, wherein the defined medium contains a serum source and is supplemented with at least one growth factor that acts through the MAPK pathway, thereby forming a population of mixed OV-CSCs, the population of mixed OV-CSCs being characterized by at least 80% of the cells in the mixed OV-CSC population expressing two or more of the biomarkers AFP, CK7, CK19, EpCAM, E-cadherin, Nanog, FoxA2 HNF4a, and ABCG2.

41. The method of claim 40, wherein the defined medium further comprises at least one growth factor that acts through the MAPK pathway.

42. The method of claim 40, the population of mixed OV-CSCs being characterized by at least 80% of the cells in the mixed OV-CSC population further expressing one or more of the biomarkers CA19-9, HER2/neu, NCAM, ganglioside CD2, estrogen receptor alpha, testosterone, TGFβR, EGFR, TAG-72, CD46, He-4, ALDH, CD133, CD44, ABCG2, nestin, and TP53.

43. The method of claim 40, the population of mixed OV-CSCs being characterized by at least 90% of the cells in the mixed OV-CSC population o expressing two or more of the biomarkers AFP, CK7, CK19, EpCAM, E-cadherin, Nanog, FoxA2 HNF4a, and ABCG2.

44. The method of claim 24, further comprising:
culturing the OV-CSC spheroids in a defined medium on an adherent substrate, wherein the defined medium contains serum and is supplemented with at least one growth factor that acts through the MAPK pathway, thereby forming a population of EMT-OV-CSCs, the population of EMT-OV-CSCs being characterized by at least 80% of the cells in the EMT-OV-CSC population expressing two or more of the biomarkers NCAM, Slug/Snail, CD24, and Twist.

45. The method of claim 44, the population of EMT-OV-CSCs being characterized by at least 80% of the cells in the EMT-OV-CSC population further expressing one or more of the biomarkers CA-125, MUC-1, CD133, Nanog, CD117, N-cadherin, CD44, and vimentin.

46. The method of claim 44, the population of EMT-OV-CSCs being characterized by at least 90% of the cells in the EMT-OV-CSC population expressing two or more of the biomarkers NCAM, Slug/Snail, CD24, and Twist.

47. The method of claim 24, wherein the defined media is any media described in Table 2.

48. The method of claim 24, wherein the defined media is any media from a combination of Table 2 and Table 3.

49. The method of claim 24, wherein the defined media is any media from a combination of Table 2, Table 3, and Table 4.

50. The method of claim 24, wherein the defined media is any media from a combination of Table 2 and Table 4.

51. The method of claim 24, wherein the growth factor is one or more of fibroblast growth factor (FGF), epidermal growth factor (EGF), or activin A.

52. The method of claim 51, wherein the FGF is basic FGF (bFGF).

53. The method of claim 24, wherein the defined medium is not supplemented with activin A.

54. The method of claim 24, wherein the defined medium is supplemented with an antagonist of activin A, in an amount effective to prevent spontaneous differentiation of ovarian carcinoma stem cells.

55. The method of claim 54, wherein the medium further comprises an antagonist of activin A, and the antagonist is follistatin or an antibody that specifically binds to activin A.

56. The method of claim 24, wherein the medium is not supplemented with an antioxidant.

57. The method of claim 56, wherein the antioxidant is superoxide dismutase, catalase, glutathione, putrescine, or β-mercaptoethanol.

58. The method of claim 24, wherein the medium is supplemented with glutathione.

59. The method of claim 27, wherein the adherent substrate is configured to adhere to, and to collect, anchorage dependent cells.

60. The method of claim 59, wherein the anchorage dependent cells are fibroblasts.

61. The method of claim 24, wherein the non-adherent substrate is an ultralow adherent polystyrene surface.

62. The method of claim 27, wherein the adherent substrate comprises a surface coated with a protein rich in RGD tripeptide motifs.

63. A population of purified OV-CSCs prepared by the method of claim 24.

64. The population of claim 63, wherein the purified OV-CSCs are in form of OV-CSC spheroids.

65. The population of claim 63, wherein the purified OV-CSCs are early OV-CSCs.

66. The population of claim 63, wherein the purified OV-CSCs are mixed OV-CSCs.

67. The population of claim 63, wherein the purified OV-CSCs are EMT-OV-CSCs.

68. An OV-CSC cell line prepared by the method of claim 24.

69. The OV-CSC cell line of claim 68, wherein the OV-CSCs are in form of OV-CSC spheroids.

70. The OV-CSC cell line of claim 68, wherein the OV-CSCs are early OV-CSCs.

71. The OV-CSC cell line of claim 68, wherein the OV-CSCs are mixed OV-CSCs.

72. The OV-CSC cell line of claim 68, wherein the OV-CSCs are EMT-OV-CSCs.

73. A method of stimulating an immune response against antigens of an ovarian carcinoma tumor in a subject in need thereof, comprising administering an immunogenic dose of an immunogenic composition comprising dendritic cells activated ex vivo by tumor antigens derived from a population of purified OV-CSCs to the subject.

74. (canceled)

75. (canceled)

76. The method of claim 30, further comprising:
culturing the mixed OV-CSCs in a defined medium on an adherent substrate, wherein the defined medium is serum free and is supplemented with at least one growth factor that acts through the MAPK pathway, thereby forming a population of early OV-CSCs, the population of early OV-CSCs being characterized by at least 80% of the cells in the early OV-CSC population expressing two or more of the biomarkers EpCAM, CD133, CD44, Nanog, Sox2, Oct3/4, CD17, and Ki-67.

77. The method of claim 76, the population of early OV-CSCs being characterized by at least 80% of the cells in the early OV-CSC population further expressing one or more of the biomarkers EpCAM, CD133, CD44, Nanog, Sox2, Oct3/4, CD17, and Ki-67.

78. The method of claim 76, the population of early OV-CSCs being characterized by at least 90% of the cells in the early OV-CSC population expressing one or more of the biomarkers EpCAM, CD133, CD44, Nanog, Sox2, Oct3/4, CD17, and Ki-67.

79. The method of claim 34, further comprising:
culturing the EMT-OV-CSCs in a defined medium on an adherent substrate, wherein the defined medium is serum free and is supplemented with at least one growth factor that acts through the MAPK pathway, thereby forming a population of early OV-CSCs, the population of early OV-CSCs being characterized by at least 80% of the cells in the early OV-CSC population expressing two or more of the biomarkers EpCAM, CD133, CD44, Nanog, Sox2, Oct3/4, CD17, and Ki-67.

80. The method of claim 79, the population of early OV-CSCs being characterized by at least 80% of the cells in the early OV-CSC population further expressing one or more of the biomarkers EpCAM, CD133, CD44, Nanog, Sox2, Oct3/4, CD17, and Ki-67.

81. The method of claim 79, the population of early OV-CSCs being characterized by at least 90% of the cells in the early OV-CSC population expressing one or more of the biomarkers EpCAM, CD133, CD44, Nanog, Sox2, Oct3/4, CD17, and Ki-67.

82. The method of claim 27, further comprising:
culturing the early OV-CSCs in a defined medium on an adherent substrate, wherein the defined medium contains a serum source and is supplemented with at least one growth factor that acts through the MAPK pathway, thereby forming a population of mixed OV-CSCs, the population of mixed OV-CSCs being characterized by at least 80% of the cells in the mixed OV-CSC population expressing two or more of the biomarkers AFP, CK7, CK19, EpCAM, E-cadherin, Nanog, FoxA2 HNF4a, and ABCG2.

83. The method of claim 82, wherein the defined medium further comprises at least one growth factor that acts through the MAPK pathway.

84. The method of claim 82, the population of mixed OV-CSCs being characterized by at least 80% of the cells in the mixed OV-CSC population further expressing one or more of the biomarkers CA19-9, HER2/neu, NCAM, ganglioside CD2, estrogen receptor alpha, testosterone, TGFβR, EGFR, TAG-72, CD46, He-4, ALDH, CD133, CD44, ABCG2, nestin, and TP53.

85. The method of claim 82, the population of mixed OV-CSCs being characterized by at least 90% of the cells in the mixed OV-CSC population o expressing two or more of the biomarkers AFP, CK7, CK19, EpCAM, E-cadherin, Nanog, FoxA2 HNF4a, and ABCG2.

86. The method of claim 34, further comprising:
culturing the EMT-OV-CSCs in a defined medium on an adherent substrate, wherein the defined medium contains a serum source and is supplemented with at least one growth factor that acts through the MAPK pathway, thereby forming a population of mixed OV-CSCs, the population of mixed OV-CSCs being characterized by at least 80% of the cells in the mixed OV-CSC population expressing two or more of the biomarkers AFP, CK7, CK19, EpCAM, E-cadherin, Nanog, FoxA2 HNF4a, and ABCG2.

87. The method of claim 86, wherein the defined medium further comprises at least one growth factor that acts through the MAPK pathway.

88. The method of claim 86, the population of mixed OV-CSCs being characterized by at least 80% of the cells in the mixed OV-CSC population further expressing one or more of the biomarkers CA19-9, HER2/neu, NCAM, ganglioside CD2, estrogen receptor alpha, testosterone, TGFβR, EGFR, TAG-72, CD46, He-4, ALDH, CD133, CD44, ABCG2, nestin, and TP53.

89. The method of claim 86, the population of mixed OV-CSCs being characterized by at least 90% of the cells in the mixed OV-CSC population o expressing two or more of the biomarkers AFP, CK7, CK19, EpCAM, E-cadherin, Nanog, FoxA2 HNF4a, and ABCG2.

90. The method of claim 27, further comprising:
culturing the early OV-CSCs in a defined medium on an adherent substrate, wherein the defined medium contains serum and is supplemented with at least one growth factor that acts through the MAPK pathway, thereby forming a population of EMT-OV-CSCs, the population of EMT-OV-CSCs being characterized by at least 80% of the cells in the EMT-OV-CSC population expressing two or more of the biomarkers NCAM, Slug/Snail, CD24, and Twist.

91. The method of claim 90, the population of EMT-OV-CSCs being characterized by at least 80% of the cells in the EMT-OV-CSC population further expressing one or more of the biomarkers CA-125, MUC-1, CD133, Nanog, CD117, N-cadherin, CD44, and vimentin.

92. The method of claim 90, the population of EMT-OV-CSCs being characterized by at least 90% of the cells in the EMT-OV-CSC population expressing two or more of the biomarkers NCAM, Slug/Snail, CD24, and Twist.

93. The method of claim 30, further comprising:
culturing the mixed OV-CSCs in a defined medium on an adherent substrate, wherein the defined medium contains serum and is supplemented with at least one growth factor that acts through the MAPK pathway, thereby forming a population of EMT-OV-CSCs, the population of EMT-OV-CSCs being characterized by at least 80% of the cells in the EMT-OV-CSC population expressing two or more of the biomarkers NCAM, Slug/Snail, CD24, and Twist.

94. The method of claim 93, the population of EMT-OV-CSCs being characterized by at least 80% of the cells in the EMT-OV-CSC population further expressing one or more of the biomarkers CA-125, MUC-1, CD133, Nanog, CD117, N-cadherin, CD44, and vimentin.

95. The method of claim 93, the population of EMT-OV-CSCs being characterized by at least 90% of the cells in the EMT-OV-CSC population expressing two or more of the biomarkers NCAM, Slug/Snail, CD24, and Twist.

96. The method of claim 30, wherein the adherent substrate is configured to adhere to, and to collect, anchorage dependent cells.

97. The method of claim 96, wherein the anchorage dependent cells are fibroblasts.

98. The method of claim 30, wherein the adherent substrate comprises a surface coated with a protein rich in RGD tripeptide motifs.

99. The method of claim 34, wherein the adherent substrate is configured to adhere to, and to collect, anchorage dependent cells.

100. The method of claim 99, wherein the anchorage dependent cells are fibroblasts.

101. The method of claim 34, wherein the adherent substrate comprises a surface coated with a protein rich in RGD tripeptide motifs.

102. A method of stimulating an immune response against antigens of an ovarian carcinoma tumor in a subject in need thereof, comprising administering an immunogenic dose of the OV-CSCs of claim 63 to the subject.

103. A method of stimulating an immune response against antigens of an ovarian carcinoma tumor in a subject in need thereof, comprising administering an immunogenic dose of the OV-CSC cell line of claim 68 to the subject.
